(19) United States
(12) Patent Application Publication
(10) Pub. No.: US 2026/0232273 A1
(43) Pub. Date: Aug. 13, 2026

(54) SYSTEM AND METHOD TO MONITOR NEURAL INTEGRITY

(71) Applicant: MEDTRONIC XOMED LLC, Jacksonville, FL (US)

(72) Inventors: Dongming Shen, Shanghai (CN); David C. Hacker, Jacksonville, FL (US); Changfeng Yang, Shanghai (CN); Chenghui Ye, Shanghai (CN)

(21) Appl. No.: 19/470,863

(22) PCT Filed: Apr. 6, 2023
(86) PCT No.: PCT/CN2023/086507

§ 371 Date: Sep. 30, 2025

(51) Int. Cl. A61B 5/00; A61B 5/294; A61B 5/388

(52) U.S. Cl. CPC A61B 5/6853; A61B 5/294; A61B 5/388

(57) ABSTRACT

A device or system and related methods are disclosed to monitor a stimulation and or evoked response of at least one nerve. The device may be positioned near a subject. A stimulation electrode assembly configured to be positioned relative to a subject is disclosed. The stimulation electrode may be a connection or self-contained component.

### CROSS-REFERENCE TO RELATED APPLICATIONS

This application includes subject matter related to International Application No. PCT/______/______ (A0009811WO01/5074X-000187-WO) and International Application No. PCT/______/______ (A0009685WO01/5074X-000189-WO). The entire disclosures of the above applications are incorporated herein by reference.

### FIELD

The present disclosure relates to system and method for activity monitoring, and particularly to neural stimulation monitoring devices and methods.

### BACKGROUND

This section provides background information related to the present disclosure which is not necessarily prior art.

During various procedures, such as various throat procedures or other procedures occurring near and/or adjacent to nerve fiber, a determination of nerve integrity or stimulation may be selected. Determining nerve integrity may include ensuring or monitoring stimulation activity along a nerve. This may include transmission of or receiving an induced signal on a nerve. In performing such integrity monitoring, an electrode or electrode containing element is connected to a nerve or nerve fiber to monitor or stimulate the nerve fiber. Monitoring of an induced signal at a single time or over a period of time can assist in determining integrity of a nerve. Various monitoring systems include the NIM-Response® 3.0 sold by Medtronic, Inc. having a place of business in Minneapolis, Minnesota. The monitor systems can include or be operated with an electrode including an APS® electrode that allows for automatic and periodic stimulation of a nerve that may be monitored by the system.

### SUMMARY

This section provides a general summary of the disclosure, and is not a comprehensive disclosure of its full scope or all of its features.

A system to sense provision of stimulation to a selected portion of a subject, such as nerve bundles or paths is disclosed. Selected systems include NIM-Response® 3.0 sold by Medtronic, Inc. that may include a selected cuff or selector for connection of electrodes to nerve modules in a wired or wireless manner. Further, a wireless stimulator assembly can be positioned adjacent to or near a nerve for stimulating the nerve and/or detecting a stimulation of the nerve. The electrodes may include an active fixation that positively connect or surround at least a portion of the nerve bundle. In the alternative, the system may provide a contact electrode that contacts the nerve and is held in place by friction or compression of surrounding tissue.

Various portions can be used to sense an evoked response in a subject. The portions may include one or more electrodes on an endotracheal (ET) tube. The ET tube may include, in addition to the electrodes, holding portions, pressure sensing portions, etc. These portions may be assist holding and/or confirming operation of the ET tube and related sensing system.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of a monitoring system and an electrode assembly;

FIG. 2 is an environmental view of an endotracheal tube, according to various embodiments;

FIG. 3 is a detailed view of a proximal end of an endotracheal tube including a pilot balloon assembly, according to various embodiments;

FIG. 4 is a detailed view of a pilot balloon for an endotracheal tube, according to various embodiments;

FIG. 5 is a detailed view of a housing for a pilot balloon, according to various embodiments;

FIG. 6 is a detailed view of a pilot balloon assembly in a first configuration, according to various embodiments;

FIG. 7 is a detailed view of a pilot balloon assembly in a second configuration, according to various embodiments;

FIG. 8 is a detailed view of a proximal end of an endotracheal tube with a pilot balloon assembly, according to various embodiments;

FIG. 9 is a perspective view of various indicators for a pilot balloon assembly, according to various embodiments;

FIG. 10 is a proximal view of an endotracheal tube and pilot balloon assembly, according to various embodiments;

FIG. 11 is a detailed view of a proximal end of an endotracheal tube and a pilot balloon assembly and a second configuration, according to various embodiments;

FIG. 12 is a schematic view of an endotracheal tube and a system to measure a pressure in a pilot balloon thereof, according to various embodiments;

FIG. 13 is a schematic view of an endotracheal tube and an assembly to measure a pressure in a pilot balloon assembly, according to various embodiments;

FIG. 14 is a schematic view of an endotracheal tube and a system to measure a pressure in a pilot balloon thereof, according to various embodiments;

FIG. 15A is a user interface view illustrating a pressure of a pilot balloon, according to various embodiments;

FIG. 15B is a user interface view illustrating a pressure of a pilot balloon, according to various embodiments;

FIG. 15C is a user interface view illustrating a pressure of a pilot balloon, according to various embodiments;

FIG. 16 is an environmental view of an endotracheal tube including a cushion assembly for an electrode contact;

FIG. 17A is a detailed view of a cushion assembly for an electrode contact, according to various embodiments;

FIG. 17B is a detailed view of a cushion for an electrode contact in a second configuration, according to various embodiments;

FIG. 17C is a detailed view taken within circle 17C of FIG. 17B;

FIG. 18A is a detailed view of a pre-assembly position of an electrode contact cushion and an endotracheal tube body;

FIG. 18B is an assembled view of an electrode contact cushion and an endotracheal tube body;

FIG. 19 is a detailed view of an assembled endotracheal tube including an electrode contact cushion;

FIG. 20A is a cross-sectional view taken along line 20A-20A of FIG. 19 with the cushion in a first configuration;

FIG. 20B is a cross-sectional view taken along line 20B-20B of FIG. 19 of an electrode contact cushion in a second configuration;

FIG. 21 is an environmental view of a surface contact return electrode, according to various embodiments;

FIG. 22 is a detailed view of a surface contact electrode, according to various embodiments;

FIG. 23 is a detailed view of leads for a surface electrode contact;

FIG. 24 is a cross-sectional view along lines 24-24 of FIG. 22;

FIG. 25 is a detailed view of a plug;

FIG. 26 is a detailed view of a socket;

FIG. 27 is a detailed view of an interconnection of a plug and a socket;

FIG. 28 is a detailed view of a distal end of an endotracheal tube with electrode contacts;

FIG. 29 is a detailed cross-sectional view taken along lines 29-29 of FIG. 28;

FIG. 30 is a detailed view of an electrode contact of an endotracheal tube, according to various embodiments;

FIG. 31 is an environmental view of an endotracheal tube, according to various embodiments;

FIG. 32 is a detailed view of a connector taken within box 32 of FIG. 31;

FIG. 33 is an environmental view of a wire assembly according to various embodiments;

FIG. 34 is an environmental view of an endotracheal tube assembly according to various embodiments;

FIG. 35 is a detailed view of the endotracheal tube of FIG. 34 within box 35;

FIG. 36 is a cross-sectional view taken along lines 36-36 in FIG. 35;

FIG. 37 is a detailed view of a printed electrode assembly, according to various embodiments;

FIG. 38 is a detailed view of a printed electrode assembly, according to various embodiments;

FIG. 39 is an environmental view of an endotracheal tube, according to various embodiments;

FIG. 40 is a detailed view of the portion of an endotracheal tube in box 40 in FIG. 39;

FIG. 41 is a detailed view of a cushion for an electrode contact, according to various embodiments;

FIG. 42 is an environmental view of an endotracheal tube including a printed flexible circuit board; and

FIG. 43 is an environmental view of a flexible circuit board.

Corresponding reference numerals indicate corresponding parts throughout the several views of the drawings.

### DETAILED DESCRIPTION

Example embodiments will now be described more fully with reference to the accompanying drawings.

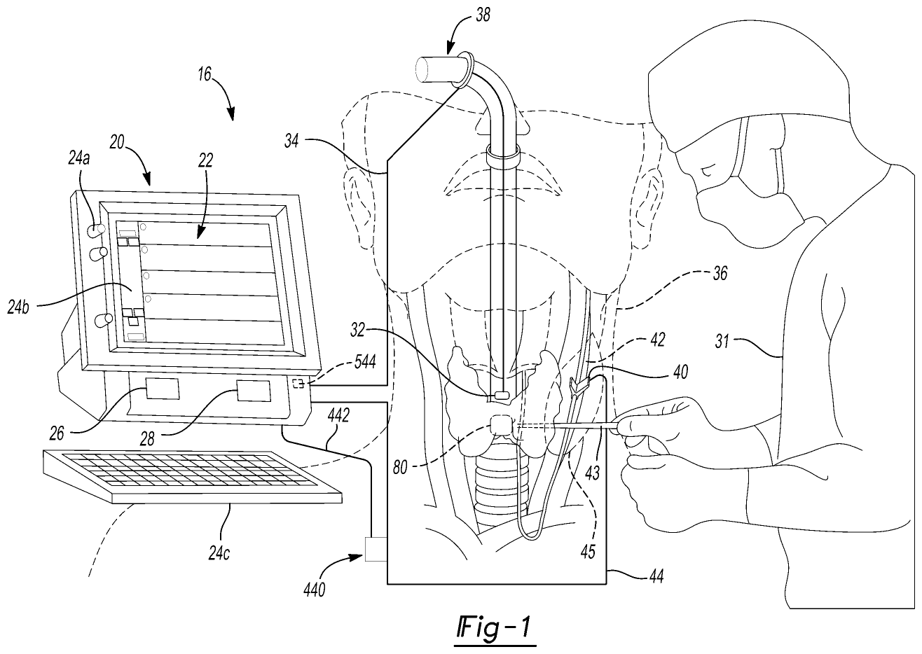

With initial reference to FIG. 1, a monitoring system 16, such as one or more nerve integrity monitoring systems, including one or more of NIM®, NIM Vital®, NIM RESPONSE®, and/or NIM-NEURO® nerve integrity monitor and component parts, may be used during a selected procedure. The monitoring system 16 may include a monitor assembly 20 that has a display screen or device 22 and one or more input devices. The monitoring system 16 may also include monitoring systems such as those disclosed in U.S. patent Ser. No. 14/678,452, filed on Apr. 3, 2015, published as U.S. Pat. App. No. 2016/0287112; U.S. Pat. Nos. 10,039,915; 9,955,882; and 10,799,152, all incorporated herein by reference. The input device may include one or more systems or structures to input commands of information such as knobs 24a, a touch screen 24b, a keyboard 24c, or other appropriate input devices. Input devices may also include audio or other tactile input devices.

The monitor assembly 20 may further include a processor 26 and a memory 28. It is understood that the processor 26 may access the memory 28 to execute instructions stored thereon or access other data on the memory 28. The memory 28 may include a physical memory, such as a spinning hard disk drive, solid state memory, or other appropriate types of memory. Further, the memory 28 may not be incorporated into the monitor assembly 20, but may be accessed by processor 26, such as via a communications network. The processor 26 may be any appropriate processor, as discussed herein, including a general-purpose processor that is operable to execute instructions for generating a selected output, as discussed further herein. The processor 26 may further include onboard memory. Accordingly, the processor 26 may execute instructions stored on memory 28, which may be a non-transitory memory, to provide an output for display on the display device 22. A user 31 may then view the display device 22 for selected purposes, as discussed further herein.

Connected with the monitor assembly 20, may be one or more stimulation or monitoring assemblies. For example, in various procedures such as a thyroidectomy or other thyroid surgeries, monitoring of a recurrent laryngeal nerve (RLN), a vagus nerve, or other appropriate nerve, in a patient 36 may be selected. Monitoring of the RLN may include a nerve monitoring esophageal tube also referred to as an endotracheal (ET) tube 38, which may have one or more conductive electrodes 32 that are in contact with selected portions of the patient 36, such as a human patient. The electrode 32 can be connected to the monitor 20, via a connection 34. It is understood, however, that the connection to the monitor 20 may also be a wireless connection where the monitor 20 receives a wireless transmitted signal from the electrode 32. It is understood, however, that various non-human patient subjects may be monitored. Non-human subjects may include robotic systems, airframes, etc.

In addition, other instruments may be connected to the monitor 20, such as electrode assemblies, including an electrode that may send or receive periodic stimulation pulses, including, according to various embodiments, a connected cuff electrode assembly 40. Other selected or appropriate electrodes include those as disclosed in U.S. Pat. Nos. 9,955,882 and 9,918,669, all incorporated herein by reference.

The stimulation electrode assemblies may be connected with a physical connection, such as a wire 44 to the monitor 20. The connection to the monitor may be wired and/or wireless, such as disclosed in U.S. Pat. No. 9,918,669, incorporated herein by reference. Thus, the monitor may be positioned at any appropriate position relative to the subject 36 and/or the user 31. The monitory 20 may be in a sterile or non-sterile location.

Other instruments may also be connected with the monitor 20 that may be used to send or receive stimulation signals to the patient to assist in determining whether nerve damage or other tissue damage has occurred or could occur. A scalpel 43 may be manipulated by the user 31, such as a human surgeon, and need not be directly connected to the monitor 20. The scalpel, however, may be connected to the monitor 20 and may be used to form an incision 45 and/or any appropriately manipulation (e.g., cut, dissection) or incision. The monitor 20 may be provided to monitor signals through or from the electrode assemblies 32 and 40 without requiring interactive stimulation or monitoring through the scalpel or other selected instruments performing a procedure on the patient 36. According to various embodiments, electrode assemblies may be connected to one or more nerves 42 to generate a stimulation to the nerve 42 at a selected rate. The rate may be selected to account for a refractory period of the nerve. Thus, a pause or period between stimulations may be selected to account for the refractory period.

The operation of the monitoring system and the use of the monitoring system 16 may be similar to the various versions and/or types of the NIM® monitoring system sold by Medtronic, Inc., including the NIM-Response® 3.0 and/or NIM VITAL® nerve monitoring system. In operation, the electrode assembly 40 may be connected with a nerve 42, as discussed further herein, and a signal may be transmitted along the connection 44 from the monitor system 20. The electrode 32 may be used to receive a signal that is transmitted through the nerve 42.

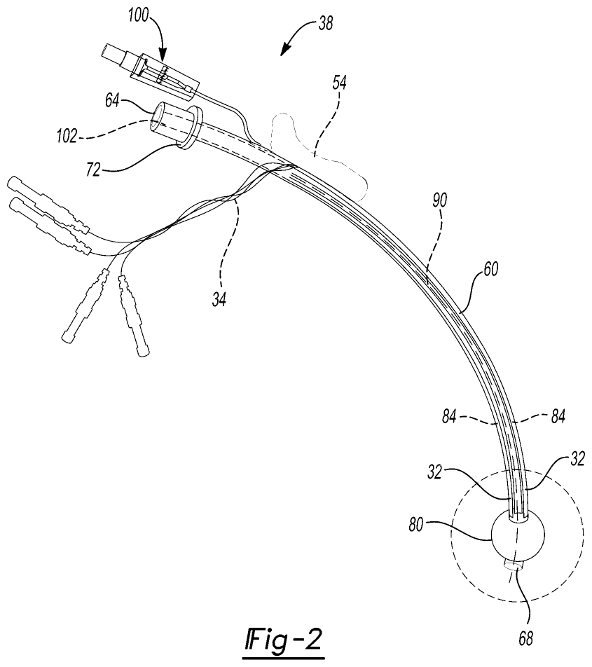

Turning reference to FIG. 2, connected with the monitor assembly 20, may be one or more stimulation or monitoring assemblies. For example, the ET tube assembly 38 that may have one or more monitoring portions, including one or more conductive electrode contacts 32. The electrode contacts 32 may be in contact with selected portions of the patient 36. The electrode contacts 32 may be connected to the monitor 20, via a connection, such as an optional wired connection (also referred to as a line or hardline) 34 or wireless connection including a wireless transmitter 54. It is understood, however, regardless of the connection to the monitor 20, a transmitted signal from the electrode contacts 32 may be made to the monitor assembly 20. An exemplary endotracheal tube may include a NIM Trivantage® monitoring tube sold by Medtronic, Inc. Exemplary endotracheal tubes may further include those disclosed in U.S. Pat. No. 9,918,669, incorporated herein by reference. It is understood, however, that the tube 38 may include portions in addition to those currently available or different from those currently available on the NIM Trivantage® monitoring tube and those discussed above.

The tube 38 may be an EMG endotracheal tube assembly 38 and a corresponding housing 60. The EMG endotracheal tube assembly 38 may include the housing 60 and an electronic assembly including the wireless transmission assembly 54 and/or the wired connection 34. The tube assembly 38 includes a tube proximal (first) end 64 and a distal (second) end 68. The distal end 64 is connected to and/or includes a connector 72, which may be connected to a pump for supplying a selected material such as a gas and/or a fluid to a patient via the tube 38. The tube 38 may be inserted in a throat of the patient 36 and the gas and/or fluid may be supplied to, for example, lungs of the patient 36.

The distal end 68 includes an inflatable portion 80 (shown in an inflated state), which may be used to seal off, for example, a trachea, to prevent any other fluid or substance from passing around the inflated portion 80 and entering the lungs. The inflatable portion 80, as discussed herein, may be inflated in a selected manner. Further, the inflation status of the inflatable portion 80 may be monitored at or near the monitor 20.

The tube assembly 38 includes the contacts 32. The contacts 32 may be in electrical connection with connections 84. The connections may be traces, wires, etc. that may be provided with and/or formed on or in the housing 60. The contacts 32 and/or the electrodes 84 may be painted or printed on and/or relative to the tube housing 60. For example, a conductive paint or ink may be applied to the tube housing 60, or a portion thereof to form the conductive portions 84. Further, the electrodes and transmission portions may include traces that are formed on flexible printed circuit boards and provided relative to the housing 60. A coating or protective layer may also be provided over the painted portions, while allowing the contacts 84 to be exposed to an external environment at selected locations or portions.

The connectors 84 may extend from the contacts 32 to the connection portion, including the wireless transmitter 54 and/or the wired connection 34. The connectors 84 may extend in parallel along the tube housing 60 and are separated as to not be in contact with each other. One or more insulation layers 90 may be applied over the connectors 84 to prevent external electrical contact with the connectors 84. Each of the insulation layers 90 may cover one or more of the connectors 84. The insulation layers 90 may be nonconductive stamps formed of nonconductive material (e.g., rubber).

As discussed above, the tube assembly 38 may be used with the monitoring assembly 20 to monitor an electrical activity signal in the patient 36. A stimulation being sensed through the contacts 32 may be provided through the electrode assembly 40 in connection with the monitor assembly 20. As discussed above, the NIM® stimulation and monitoring system sold by Medtronic, Inc. may be provided to sense at or stimulate the nerve. Upon sensing the stimulation from the nerve (including, for example, an EMG response of a muscle and/or electrical activity related thereto), the monitoring system 20 may monitor the EMG signal after determining a baseline and to determine the baseline. Further, the user or other appropriate individual may observe the monitoring system 20, such as the display 22, to ensure integrity of nerves during a surgical procedure. Procedures may include a throat or thyroid removal procedure, as discussed above.

The one or multiple contacts 32 can be provided around the tube assembly 38 for various purposes. In various embodiments, the multiple electrodes and/or contacts therefore may be used for differentiating between left and right nerves, differentiating between different nerves and nerve branches, compensating for users placing the tube contacts in a variety of depths and/or axial positions relative to the anatomy. The multiple electrodes may be placed axially along the tube to allow for measurements at a distance from a selected location as well. Referential recording electrodes may also be placed a distance from other recording electrodes to minimize noise and interference. Upon movement of the contacts 32 relative to the patient 36 (including internal tissue, such as muscle that may include vocal fold in a human larynx), the signal to the monitoring system 20 may change. Change of the signal to the monitoring system 20 may be interpreted or possibly interpreted as an injury to the nerve 56. The system 20, upon determining a change or sensing a change in the received stimulation, may provide an indication to the user 30 that an injury has occurred and that the procedure should be stopped. If the signal to the monitoring system 20, however, changes only due to movement, whether intentional or unintentional, of the tube assembly 40 then no injury has occurred, as disclosed in U.S. Pat. No. 10,799,152, incorporated herein by reference.

In various embodiments, the ET tube 38 may include various features and portions including those discussed herein. With continuing reference to FIG. 1 and additional reference to FIGS. 3 through 7, the ET tube 38 includes a pilot balloon portion or assembly 100. The pilot balloon assembly 100 may be used to determine an inflation status of the inflatable portion 80. As discussed above, the inflatable portion 80 may be used to limit or eliminate a space or passage passed the ET tube 38 other than a cannula or passage 102 formed through the ET tube 38. The cannular 102 may be formed or defined by an internal wall of the ET tube 38. The cannular 102 may allow for passage of material into the subject. As the cannula or passage 102 allows a limited passage of material, such as gas, into the subject 36, the inflatable portion 80 may be provided to ensure that no other material passes into the subject 36. Therefore, during a selected procedure, it may be desirable or selected to ensure an inflation status of the inflatable portion 80 to ensure the limitation or elimination of a passage passed the inflatable portion 80.

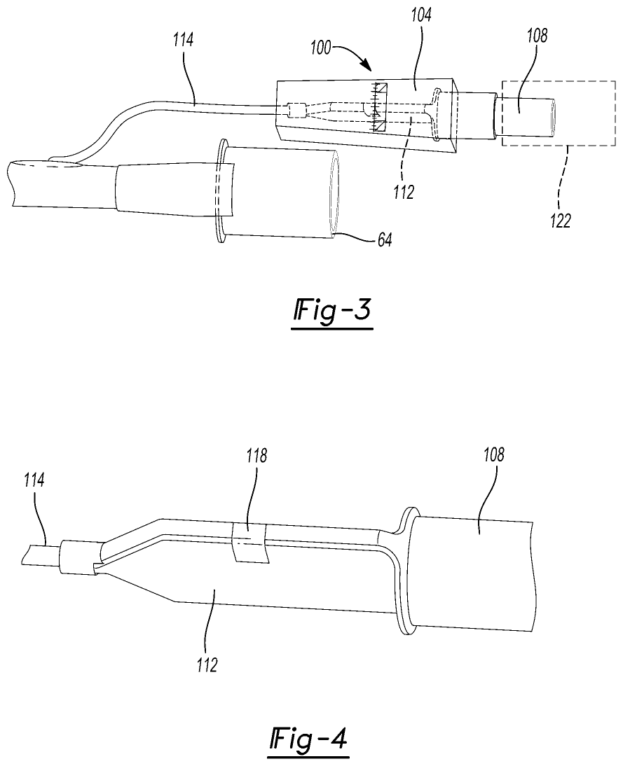

With initial reference to FIGS. 3 and 4, the pilot balloon assembly 100 includes a pilot balloon casing or holder 104. The pilot balloon assembly may include a pilot balloon connector 108 and a pilot balloon portion 112. The pilot balloon portion 112 may expand within the housing 104 when pressure is provided within the expandable portion 80. The pilot balloon 112 may be inflated to a selected degree based upon a volume and pressure within the inflatable portion 80 after being sealed or filled a selected amount through the connector 108. For example, during use of the ET tube 38, the inflatable portion 80 may be filled via the pilot balloon assembly 100 such as providing a volume of gas through the connector 108, through the pilot balloon at 112, and the inflatable portion connector 114 to the inflatable portion 80. Once the inflatable portion 80 is inflated to a selected degree, such as based upon a selected pressure, the connector 108 may be sealed. The pilot balloon 112, therefore, may also be filled to a selected degree based upon a pressure within the system between the inflatable portion 80 and the pilot balloon assembly 100. The pilot balloon 112, according to various embodiments as discussed herein, may be used to determine an inflation status of the inflatable portion 80 by various techniques. In other words, the pilot balloon assembly 100 may be used, such as by the user 31, to determine and/or confirm the status of the inflatable portion 80. Thus, the operation of the ET tube 38 may be used and the inflatable portion 80 operational to seal a passage (e.g., an esophagus) in the subject 36.

According to various embodiments, as illustrated in FIGS. 3 and 4, the pilot balloon 112 is positioned within the housing or shell 104. The pilot balloon 112 may be filled from a selected volume with the connector 108 of the pilot balloon assembly 100. A fluid, such as a gas (e.g., sterile air) initially passed through the pilot balloon assembly 100 and fill the inflatable portion 80, as discussed above. Once the inflatable portion 80 is filled to an appropriate amount, the flow of fluid through the pilot balloon 112 may be stopped. The connector 108 may be closed in an appropriate manner. For example, the connector 108 may be sealed such as with a one-way valve. In this manner, when the connector 108 is sealed the pressure experienced at the pilot balloon 112 is due to the volume of gas in the system, including in the inflatable portion 80 and through the connector 114.

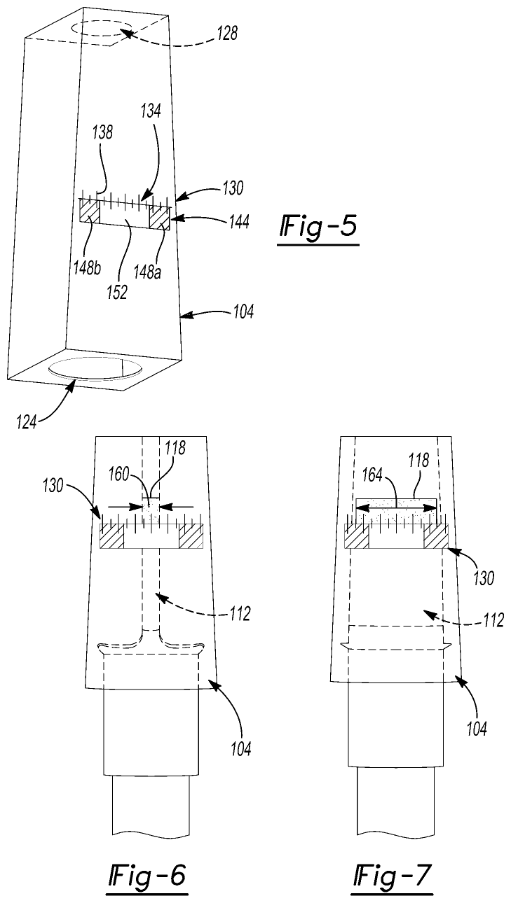

At a selected pressure, the pilot balloon at 112 may fill a selected portion of the shell 104, as illustrated in FIGS. 5 and 6. As illustrated in the figures, and with particular reference to FIG. 5, the shell 104 may be formed of a material that may allow the user 31 to view the balloon 112, such as a translucent or transparent material. In other words, the shell 104 may be formed of the selected material that allows for viewing of at least a portion of the pilot balloon 112 within the shall 104.

The shell 104 may include an internal volume in which the pilot balloon 112 is placed. The shell 104 may include openings at selected portions, such as a first opening 124 at a first end and a second opening 128 at a second end. The pilot balloon 112 may be assembled within the shall 104 in any appropriate manner. For example, the pilot balloon 112 may be assembled within the shell 104 and/or the shell 104 may be snap fit over the pilot balloon 112. Regardless, the pilot balloon 112 may expand within the shell 104 and provide an indication therein of a status of the inflation of the inflatable portion 80.

In any appropriate manner, the shell 104 may be generally rigid relative to the pilot balloon 112. Thus, the pilot balloon 112 may expand against and fill an internal volume of the shell 104. This allows the user 31 to gage a volume within the pilot balloon 112, as discussed herein.

In various embodiments, for example, an indicator region 130 may be provided on the shell 104. The indicator region 130 may include one or more indicator portions. In various embodiments, a scale indicator 134 may include a plurality of demarcations 138. Demarcations 138 may include an objective scale, such as indicating a length including millimeters (mm). In various embodiments, the scale or demarcations may only identify or indicate a relative scale, such as relative to the size of the case or shell 104.

In various embodiments, the scale indicator 134 may also or alternatively include a block indication 144. The block indication 144 may include different block portions, such as identified by colors, to indicate various amounts of inflation of the pilot balloon 112. The block indications may include an indication of appropriate inflation which may include one or more block portions 148a, 148b. A second block indication 152 may include a cautionary inflation indication.

Accordingly, the selected scale or demarcations 130 may be provided to allow a user, such as user 31, to view the pilot balloon 112 within the shell 104. Thus, the user 31 can identify efficiently the status of the inflatable portion 80 as a size of the pilot balloon 112 within the shell 104 is indicative of the status of the inflatable portion 80. The indicator scale 130 may be usable by any appropriate individual to view the pilot balloon 112. Further, the scale may be calibrated during the procedure to determine what change and/or indication would require or be indicative of possible actions by the user 31.

With particular reference to FIGS. 6 and 7, the status of the inflatable portion 80 may be understood by the user 31 by viewing the indicator 118 on the balloon 112. As illustrated in FIG. 6, the indicator 118 includes a first dimension 160. The indicator 118 may be viewed by the user 31 relative to the marking region 130. As illustrated in FIG. 6, the user 31 may view that the pilot balloon 112 is substantially deflated. Therefore, the user may understand that the inflatable portion 80 is substantially deflated or has a very low pressure therein.

During use, such as when initially inflating the inflatable portion 80, the user 31 may inflate the inflatable portion 80 and view the indicator 118 to have the second dimension 164, as illustrated in FIG. 7. That is, the user 31 may inflate the inflatable portion 80 an appropriate amount when the user 31 views the region 130 relative to the indicator 118 in the second dimension 164.

During use of the ET tube 38, therefore, the user 31 may continue to view the indicator 118 relative to the marking region 130. If the user 31 notices a change relative thereto, the user 31 may understand a change in the inflation status of the inflatable portion 80. For example, if the size of the indicator 118 relative to the marking region 130 shrinks the user 31 may understand that the inflatable region 80 has leaked or no longer has a selected pressure therein. The user 31 may then determine whether a change or reinflation should be performed. Further, the user 31 may view the indicator 118 relative to the scale region 130 and notice an increase in the indicator 118 relative thereto to understand an increase in pressure has occurred. Accordingly, during use, the user 31 may understand a change in a pressure or inflation status of the inflatable portion 80 by viewing the indicator 118 relative to the indication region 130 on the shell 104.

In this way, the pilot balloon 112 may inflate and deflate relative to the shell 104. The balloon 112 may be formed of a flexible or elastic material such as polyvinyl chloride (PVC). Alternatively, the balloon 112 may be formed of an inelastic material. The balloon 112, however, may change inflation status relative to the shell 104. The shell 104 is generally rigid and will not change size based on pressure exerted by the balloon 112 during use of the ET tube 38. Thus, the indication 130 on the shell may be used to determine and/or view a change inflation status of the balloon 112.

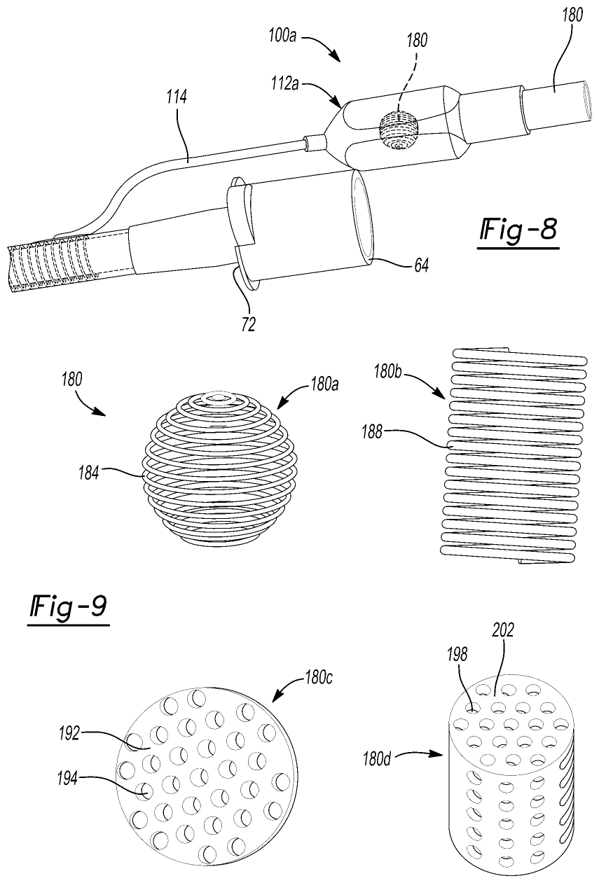

Turning reference to FIG. 8 through 11, and indicator or pilot balloon assembly 100a is illustrated. The indicator assembly 100a may include portions similar to the pilot balloon assembly 100, discussed above. Generally, the indicator assembly 100a may also be used to inflate the inflatable portion 80 by connecting to connector 108 and passing a volume of gas through the indicator assembly 100a and through the connection tube 114 to the inflatable portion 80. The indicator assembly 100a may include a pilot balloon 112a. Within the pilot balloon 112a may be an indicator 180. The pilot balloon 112a may allow viewing of an interior space or volume of the pilot balloon 112a. The pilot balloon 112 a may be formed up a selected material, such as a polymer including PVC. The selected material of the pilot balloon 112a, however, is generally translucent or transparent.

Similar to the processes discussed above, the inflatable portion 80 may be inflated with the select volume and/or to a selected pressure. Thereafter, the inflatable portion 80 may be selected to be maintained in a selected state (e.g., pressure or volume) as long as a leak in the system does not occur and the inflatable portion 80 maintains a selected inflation pressure by closing the connector 108, such as with a one-way valve. The indicator 180 may be viewed within the pilot balloon 112a to assistant determining an inflation status of the inflatable portion 80. At a selected inflation, the pilot balloon 112a may include an internal volume great enough to allow the indicator 180 to move within the pilot balloon 112a. User 31 may review the status of the indicator 180 within the pilot balloon 112a when the inflatable portion 80 is initially inflated to a selected amount.

The indicator 180 may be provided in any appropriate manner, as exemplarily illustrated in FIG. 9, the indicator 180 is generally a three-dimensional object that may be movable within the internal volume of the pilot balloon 112a when the pilot balloon 112 is inflated to a selected amount. In various embodiments, indicator 180 may be one or more of the indicators as illustrated in FIG. 9. For example, an first indicator 180a may include a wire 184 that is wound into a spring ball or sphere shape. In a similar manner, an indicator 180b may include a wire 188 that is wound into a cylindrical shape. Other configurations may include an indicator 180c that includes a three-dimensional shape having an exterior surface 192 with one or a plurality of bores 194 formed through the surface 192. An indicator 180d may include a substantially cylindrical surface 198 through which a plurality of bores 202 may be formed. It is understood that any of the indicators 180 may also be substantially solid members having substantially uninterrupted or continuous exterior surfaces. According to the various embodiments, the indicators 180 may be provided in the pilot balloon 112a to indicate an inflation status of the pilot balloon 112a which may be interpreted as a status of the inflatable portion 80.

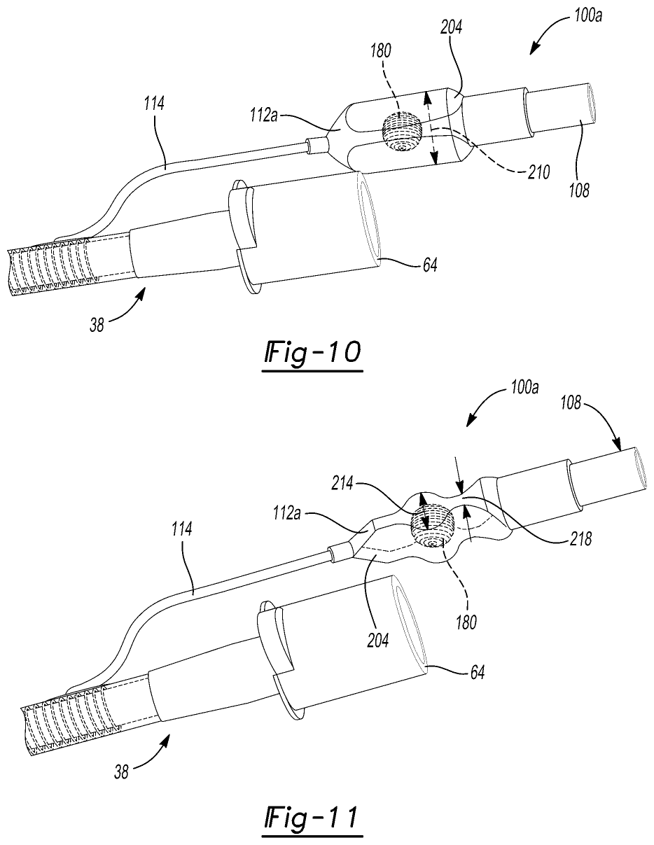

With continuing reference to FIGS. 8 and 9 and additional reference to FIGS. 10 and 11, the pilot balloon assembly 100a is illustrated in use. As discussed above, the inflatable portion 80 may be inflated to a selected amount (e.g., pressure or volume). The pilot balloon 112a may then be inflated to a selected amount and sealed, such as with a one-way valve in the connector 108. To achieve this, the pilot balloon 112a includes an external wall 204 that expands to a selected dimension and forms an internal dimension 210. The internal dimension 210 may be any appropriate dimension and it may define any appropriate shape within the pilot balloon 112a. The internal dimension 210 may be selected to allow for the indicator 180 to move within the internal volume of the pilot balloon 112a, as illustrated in FIG. 10. Movement of the indicator 180 within the pilot balloon 112a may be a visual and/or tactile indication to the user 31 that the pilot balloon 112a is inflated to at least a selected amount. This may, in turn, provide an indication to the user 31 that the inflatable portion 80 is appropriately or selectively inflated.

With reference to FIG. 11, if a leak or deflation of the inflatable portion 80 occurs or leaking any portion of the system, the pilot balloon 112a may be deflated. The external wall 204 of the pilot balloon 112a may collapse due to a formation and/or material of the pilot balloon 112a. Once collapsed, the internal dimensions of the pilot balloon 112a may become smaller or different than the initial or inflated internal dimension 210. For example, a first dimension 214 may exist substantially adjacent to and/or in contact with of the indicator 180. A different or smaller internal dimension 218 may also be formed away from the indicator 180.

Regardless of the specific internal dimension, the internal dimensions 214, 218 may cause the pilot balloon 112a to capture or engage the indicator 180. If the indicator 180 is engaged by the pilot balloon 112a it may generally cause the indicator 180 to be immovable or substantially reduced in the freedom of movement within the pilot balloon 112a. Thus, the user 31 may understand that the indicator 180 is limited in movement and that a leak is possible in that the inflation system.

Thus, as illustrated in FIG. 10, the indicator 180 may move within the internal volume of the pilot balloon 112a due to the inflated dimension 210 of the pilot balloon 112a. The user 31 may view or feel this movement and understand that the inflatable portion 80 is inflated to a selected amount. As illustrated in FIG. 11, however, the pilot balloon 112a may capture or engage the indicator 180. The user 31 may view or feel the lack of movement and understand that the inflatable portion 80 is not inflated to a selected amount and there may be a leak in the inflation system.

The indicator balloon 112a may be formed of a material, as noted above, that may be elastically expanded. When under a selected pressure, such as a pressure to maintain a selected inflation of the inflatable portion 80, the pilot balloon 112a may achieve the internal dimension 210. The wall of the chamber holding indicator 180 maybe an external wall or any appropriate wall of the balloon 112a. When a selected pressure or volume of gas is not within the balloon 112a the material of the balloon 112a may elastically contract to engage the indicator 180, as illustrated in FIG. 11. Therefore, the pilot balloon 112a may be formed of a material that may expand under a selected pressure and contract to engage the indicator 180 when the pressure is not present.

Again, the pilot balloon 112a may include the external wall 204 or the wall 204 may be a wall of an expandable material that is held within a selected case. Regardless, the indicator 180 may be engaged when the pressure is not achieved within the balloon 112a to provide a visual and/or tactile or direct feedback to the user 31 of a status (e.g., inflated or uninflated) of the inflatable portion 80.

In addition to or alternative to the above indications, according to various embodiments, a pressure within the pilot balloon may be displayed on the monitoring system 20. For example, as illustrated in FIG. 12, a pilot balloon assembly 110b may include a pilot balloon 112b and the connector 108. The pilot balloon assembly 110b may be connected with the ET tube 38 in a manner similar to that discussed above. Accordingly, at an appropriate time, the inflatable portion 80 may be inflated with an inflation assembly and may be disconnected from the connector 108. The connector 108, however, is connected to the pilot balloon 112b. A measurement of pressure within the pilot balloon 112b may, therefore, be made through the connector 108 and/or at the pilot balloon 112b.

In various embodiments, for example, a conduit 250 may be connected to the connector 108. The conduit 250 may be any appropriate conduit that may allow for a fluid connection between the connector 108 and an interface box 254. The conduit 250, for example, may be a hollow tube that is connected to the connector 108 and also connected to the interface box 254. The conduit 250, therefore, may allow for a transfer of fluid from the connector 108 to the interface box 254. This also allows a pressure to be directly sensed at the connector 108 through the conduit 250. The conduit 250, therefore, may also generally be rigid or non-expanding under the normal operating pressures of the pilot balloon 112b.

As the conduit 250 is connected to the interface 254, the interface 254 may include a pressure sensor 258. The pressure sensor 258 may be any appropriate pressure sensor that may sense of pressure in the conduit 250. The pressure sensor 258, therefore, may be a Microsensor MPM280 series of pressure sensors. The pressure may be calculated as a pressure in the interface box 254 and a signal may then be transferred or transmitted to the display assembly 20, via a selected connection 262. The connection 262 may be wired connection and/or may be a wireless connection. Further, the signal transmitted with the connection 262 may be a raw signal (e.g., analog or unprocessed) from the pressure sensor 258 so that the monitoring assembly 20 may determine the pressure based upon the signal transmitted from the connection 262 of the pressure sensor 258. Alternatively, the pressure sensor 258 may determine a pressure and the signal transmitted may be a signal of the determined pressure. The pressure determination may be any appropriate pressure, such as millimeters of mercury, pounds per square inch, etc.

A representation of the pressure may be displayed with the monitoring system 20. The displayed pressure may be an absolute pressure, such as millimeters of mercury, pounds per square inch, or the like. In addition, and/or alternatively, the pressure may be a relative pressure. The pressure may be displayed in a numerical value, such as a numerical pressure value 268. Alternatively, and/or additionally, the pressure value may be displayed in a graphical manner with a graphical representation 272. The graphical representation 272 may illustrate various selected boundaries such as an upper boundary 274 and a lower boundary 276. The pressure may be displayed over time as a pressure line 278. Therefore, a real time display of the pressure may be displayed numerically and/or graphically. Further, a representation of the pressure over time may also be displayed, such as with a plurality of specific numerical values or with the graph line 278. The monitor assembly to 20 may also display the other appropriate information, such as the EMG sensed signal 282. Thus, the user 31 may view the display of the monitoring assembly 20 to understand all measured or sensed parameters regarding the subject 36.

Therefore, a direct measured pressure in the pilot balloon 112b may be presented to the user 31. The user 31 may view the monitoring assembly 20 to make a determination of the pressure value that is sensed in the pilot balloon 112b. The user 31 may then identify or determine whether the pressure in the pilot balloon is appropriate for the selected procedure. Additionally, the user 31 may identify upper or lower limits and the pressure graph or value may be displayed relative to those, as discussed further herein.

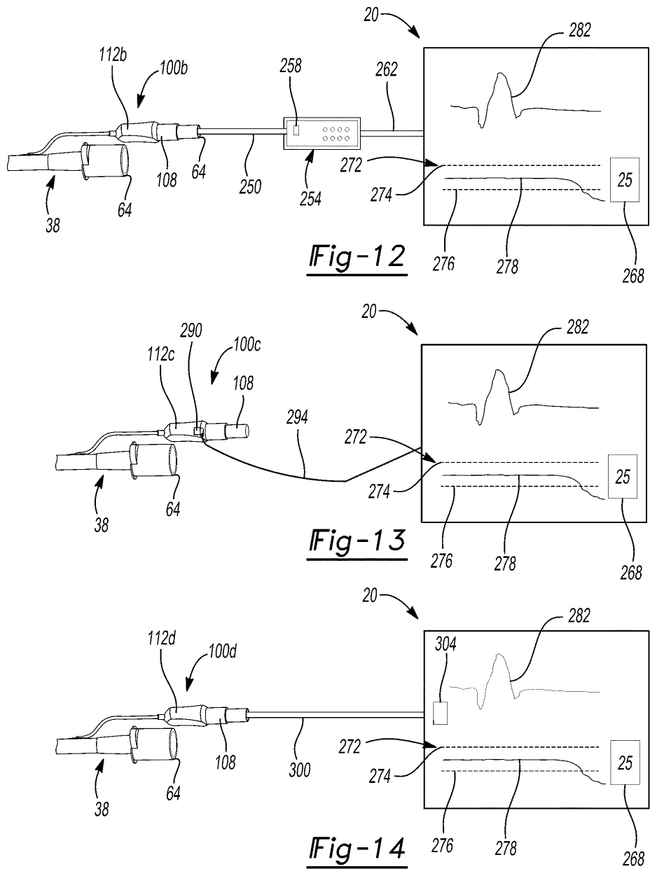

Turning reference to FIG. 13, a pilot assembly 100c is illustrated. The ET tube 38 may include the pilot assembly 100c including a pilot balloon 112c. The pilot balloon 112c may have a pressure that relates to the inflatable portion 80, as discussed above. As discussed above, the connector 108 may be used to fill the inflatable portion 80 to a selected pressure and then may be sealed, such as with a one-way valve. Therefore, the connector 108 may be sealed and the pressure within the pilot balloon 112c may relate to the pressure of the inflatable portion 80. A pressure sensor 290 may be placed in or adjacent to the pilot balloon 112c. The pressure sensor 290 may measure or sense the pressure within the pilot balloon 112c. A signal based upon the sensed pressure may be transmitted, via a connection 294, to the monitoring assembly 20. The signal transmitted with the connection 294 may be a calculated pressure based upon the pressure sensor 290 and/or a signal sensed with the pressure sensor 290 to allow for calculation of pressure with the monitoring assembly 20. Further, the connection 294 may be any appropriate type of connection such of a wired connection, wireless connection, or combination thereof. Further, the pressure sensor 290 may be any appropriate pressure sensor, such as a solid state pressure sensor including a Microsensor MPM280 series of pressure sensors.

The pressure sensor 290 measures the pressure in the pilot balloon 112c that relates to the pressure of the inflatable portion 80. The position of the pressure sensor on or in the pilot balloon 112c eliminates a fluid connection to the inflatable portion 80. As the connector 108 may be sealed, the signal connection 294 may be a digital or electrical signal connection such that the connector 108 may be maintained substantially sealed. Further, the pressure sensor 290 may be positioned within the pilot balloon 112c to reduce error due to transfer of pressure or increasing the volume of the system.

Nevertheless, the pressure sensor 294 may allow for a display of the sensed pressure on the monitoring assembly 20. Again, the graphical representation 272 may display the representation of the pressure on the monitoring assembly 20 and/or the numerical representation 268 may be displayed. As discussed above, the graphic representation may also include illustrations of selected limits 274, 276 and a graphical line representation of the sentenced pressure over time 278. Additionally, other information may be displayed such as the EMG signal 282. Again, this allows the user 31 to view a representation of the pressure sentenced in the pilot balloon 112c to make a determination of whether the pressure or status of the inflatable portion 80 is selected or appropriate.

With reference to FIG. 14, a pilot balloon assembly 100d is illustrated. The pilot balloon assembly 100d may include the pilot balloon 112d and the connector 108 that is connected to the inflatable portion 80 of the ET tube 38, in a manner similar to that discussed above. Thus, the pilot balloon 112d may be filled to the pressure of the inflatable portion 80 to determine if the inflatable portion 80 is filled or pressurized to the selected amount. The connector 108 may be connected to a measuring or transfer conduit 300 that may allow transfer of pressure, such as via a fluid, to a pressure sensor 304 at the monitoring assembly 20. The conduit 300 may include a tubing that allows for a fluid passage such that a direct measurement of the pressure within the pilot balloon 112d may be made at the connector 108.

The pressure sensor 304 at the monitoring assembly 20 may be an appropriate pressure sensor. Further, the pressure sensor 304 sensed at the monitoring assembly may be the pressure based upon the pressure in that the conduit 300. Thus, the monitoring assembly 20 may include a fluid connection to the pilot balloon 112d to allow for determination of the pressure therein.

The monitoring assembly 20 may then include the various pressure display portions such as the numerical display 268 and the graphical display 272. As also discussed above, the graphical display may include an illustration of selected limits 274, 276 and a graphical representation of the pressure 278. The monitoring assembly 20 may also illustrate other information such as the EMG signal 282. Thus, the monitoring assembly 20 may display a measured pressure within the pilot balloon 112d due to the conduit 300. The conduit 300 may include a fluid conduit to allow for a direct measurement of pressure within the pilot balloon 112d due to the transfer of pressure to the pressure sensor 304 at the monitoring assembly 20.

The pressure sensor 304 may be any appropriate pressure sensor, such as those noted above. The pressure sensor 304 may be incorporated into the monitory assembly 20 in any appropriate manner. Further, the signal from the pressure sensor 304 may be any appropriate signal and used to determine and display the pressure. Thus, the pressure sensor may be incorporated into the monitoring assembly to assist in reducing cost of various components that may be disposable, such as the ET tube 38, or other instruments.

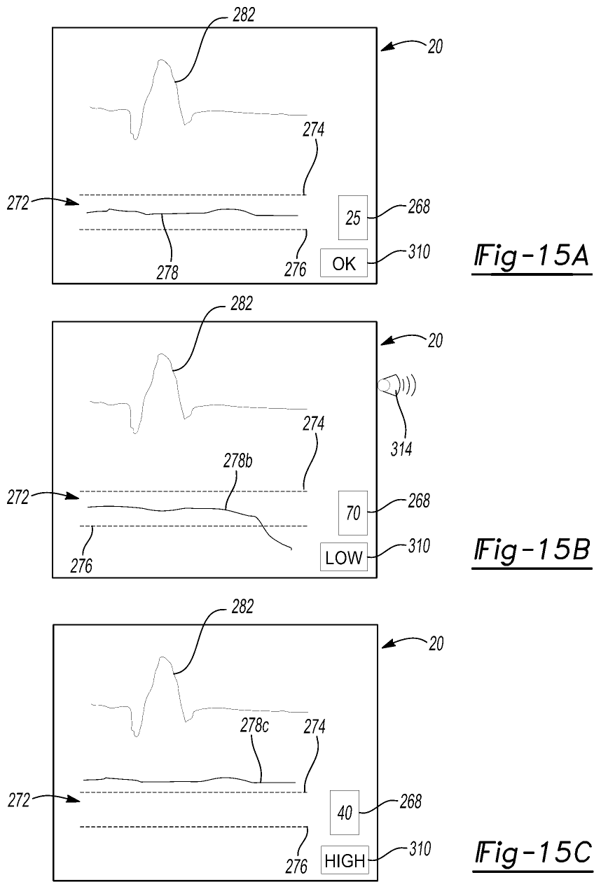

With continuing reference to FIGS. 12 through 14, and additional reference to FIGS. 15A, 15B, and 15C, the monitoring assembly 20 may include selective feedback to the user 31 based upon the measured or sensed pressure in the pilot balloon 112, according to various embodiments. As discussed above, the user 31 may identify selected ranges for the measured pressure. As illustrated in FIG. 15A, the graphical representation 272 may graphically represent selected limits, such as the upper limit 274 and the lower limit 276. The pressure graph 278 is between the limit lines and, therefore, the measured pressure is within the selected or input limits. The numerical value 268 may also be within the selected range. In such an instance, a graphical representation of the status 310 may be displayed on the monitoring assembly 20. The status display 310 may be a selected color, such as green, and include a word such as “OK”.

Turning reference to FIG. 15B, therein the graphical representation of the pressure 278 includes a low portion 278b that is below the lower limit 276. The numerical value 268 may also be below the lower limits and may include a graphical change relative to the limit. Further, a visible or graphical status indicator 310 may also indicate a change in color, such as change to red. The user 31 may set that as a low pressure or as a warning pressure and may also provide that an audible output be made, such as an alarm from a speaker 314. Thus, the user 31 may be cautioned or identified that the pressure sensed with the monitoring assembly 20 is below the preset lower limit. This may allow the user 31 time or an opportunity to assess the system, including the ET tube 38, to determine a selected action. As discussed above, the user may select the limits and the user may also select the appropriate outputs based upon the passage of the limits, such as a color change, graphical change, audible signal, or the like.

Turning reference to FIG. 15C, the monitoring assembly 20 may illustrate the events, such as with the graphical representation 278c as being above the selected upper limit 274. Similarly, the numerical value 268 may illustrate the same and a graphical representation 310 may also be provided to illustrate the above upper limit pressure. The user 31 may select to that being higher than the selected upper limit is cautionary and may not require an audible output. Nevertheless, of the display may change in color, or other features to provide an indication to the user 31 that the pressure is measured above the preselected upper limit. Additional visual notices may be provided to the user 31.

Accordingly, pressure monitoring based upon a pressure sensor at the pilot balloon 112 may be made according to various embodiments. The pressure sensor may sense a pressure and provide a signal based upon the sensed pressure. The signal may be analyzed to determine a pressure value that may be displayed with the monitoring assembly 20. The pressure value may be a direct pressure value or may be a representative relative pressure of the pilot balloon. Nevertheless, the pressure may be sensed with the selected sensor and a value and/or graphical representation of the sensed pressure may be displayed for viewing by the user 31. Further, various outputs may be provided based upon the sensed pressures, as discussed above. The outputs may be viewable, heard, and/or felt by the user 31.

As discussed above, the ET tube 38 may be positioned within the subject 36 for various procedures. The ET tube 38 includes the electrodes 32 that may be positioned near various anatomy of the subject 36 to sense an evoked response therein. As also discussed above, the ET tube 38 may be positioned relative to other portions of the subject 36 and/or other subjects. Nevertheless, the electrode contact 32 are generally positioned relative to a selected portion of the subject 36 to receive a signal or sense a signal.

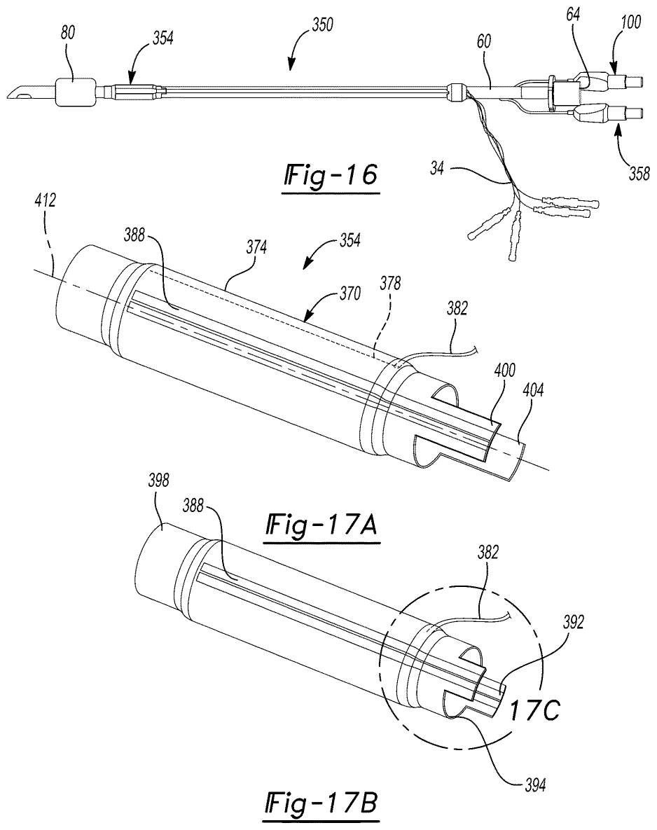

Turning reference to FIG. 16, an ET tube 350 is illustrated. The ET tube 350 may be substantially similar to the ET tube 38, discussed above, with differences discussed herein. The ET tube 350 includes the connections 34 that may extend from the distal region of the ET tube 350, generally near the contact 354, to the monitor assembly 20. As discussed herein, the contact assembly 354 may be the contact 32, as discussed above, with the various features as discussed herein. The ET tube 350 further includes the expandable portion 80 that may be inflated through a connection, such as the pilot assembly 100.

The ET tube 350 may further include a second inflation connection 358. The inflation connection 358 may extend at or near the contact assembly 354, as discussed herein. Each of the inflation connections 100, 358 may be used to pass a selected volume of gas to the related inflatable portions, such as the expandable portion 80 and near the electrode assembly 354. Lumens relative to the body 60 of the ET tube 350 transfer the gas to the selected regions.

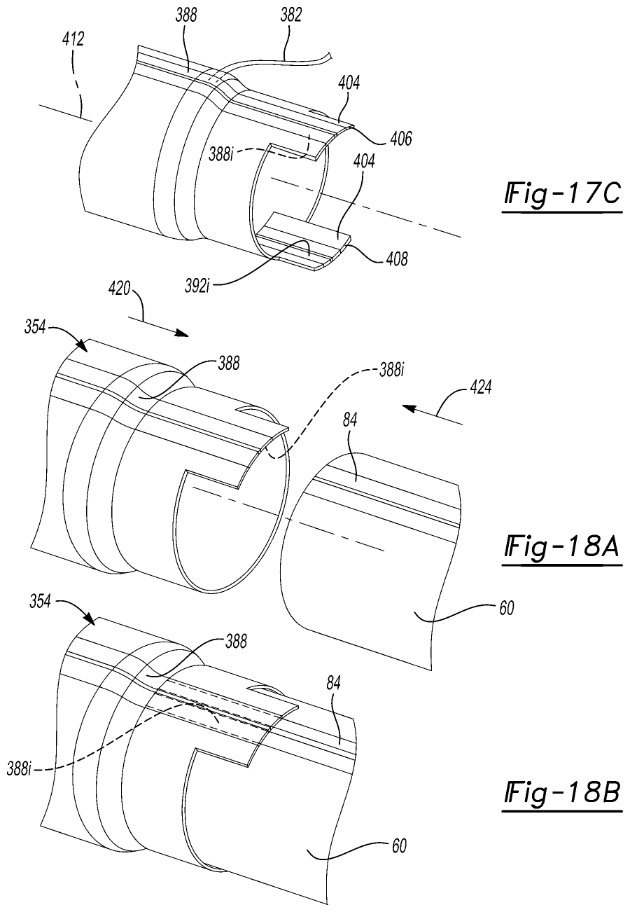

With continuing reference to FIG. 16 and additional reference to FIGS. 17A, 17B, and 17C, the electrode assembly 354 will be described in greater detail. The electrode assembly 354 may include a sleeve or cuff 370 having an exterior wall 374. The cuff 370 may further include an internal bladder that may be an annular bladder 378. The bladder 378 may be filled via the cannula 382 that extends from the second inflation connection 358. Therefore, a volume of gas may be passed to the bladder 378 to fill it to a selected amount. The connection 358 may then be sealed, similar to the pilot balloon assembly 100, to maintain a selected volume of gas and/or pressure within the bladder 378. As discussed herein, the bladder 378 may allow expansion of the external surface 374 of the sleeve 370.

The electrode assembly 354 further includes one or more contact portions, such as a first contact portion 388 and a second contact portion 392. The contact portions may extend along of the sleeve 370 a selected distance such as from a proximal end 394 toward a distal end 398 of the sleeve. The contact portions 388, 392 may extend all or only a portion of the distance along the sleeve 370. The contact portions may include a single contact or a dual contact, based upon the configuration of the electrode assembly 354.

The contacts 388, 392 may be formed on the exterior surface 374 of the sleeve 370 in any appropriate manner. For example, in the contacts 398, 392 may be painted on to the exterior surface was selected conductive material. Additional or alternative application techniques may include pad printing or screen printing. The contact 388, 392 may also be formed as a flexible printed circuit and formed or placed on the surface 374 of the sleeve 370. Other appropriate processes may also be used to form the contact 388, 392, as is generally known in the art including a coating process.

The sleeve 370 may be formed with a plurality of tabs, such as a first tab 400 and a second tablet 404. The tabs 400, 404 may be provided in any appropriate number and may relate to the number of contacts on the electrode assembly 354. The tabs 400, 404 may have the contacts 388, 392, or at least a portion thereof, formed on an exterior surface of the tabs 400, 404. The tabs 400, 404 may then be folded toward the distal end 398 of the sleeve 370 such that the contact portions extend around a proximal edge 406 of the first tab 404 and a proximal edge 408 of the second tab 404. Further, the contact portions may then also be formed on an interior surface or diameter of the sleeve 370 due to the folding of the tabs 400, 404, as illustrated in FIG. 17C. The second contact 392 may have an internal portion 392i, as illustrated in FIG. 17C. Similarly, the contact portion 388 may also have an internal portion within the sleeve 370. Thus, an electrical contact with the internal portion will also allow for an electrical continuity with the exterior portion, as illustrated in FIGS. 17A and 17B.

The bladder 378 may be formed between the exterior surface 374 and an interior surface of the sleeve 370. The bladder 378, however may be formed or provided at any appropriate position on the sleeve 370 to move the contacts 388, 392 as discussed herein. Therefore, the bladder 378 may be inflated to expand or remove the exterior surface 374 and, therefore, the contact 388, 392 away from a central axis 412 of the sleeve 370. As discussed herein, therefore, this may allow the contacts 388, 392 to be positioned in selected contacts with portions of the subject 36.

The electrode assembly 354 may, therefore, be connected with the tube body 60. The traces or contact connections 84 may extend along the body 60 of the ET tube 350. The traces or contacts 84 may connect with the contacts 388, 392 of the electrode assembly 354 when the electrode assembly 354 is assembled on to the tube body 60. For example, is illustrated in FIG. 18A, the electrode assembly 354 may be moved generally in the direction of arrow 420. The tube 60 may also and/or alternatively be moved in the direction of arrow 424. The traces 84 may, therefore, contact at least the internal portions 392i, 388i of the contacts 388, 392. Therefore, electrical continuity may be provided to the electrode assembly 354 by the connection or contact of the traces 84 with the internal portions 392i, 388i of the respective contacts, such as the internal portion 388i of the contact 388 is illustrated in FIG. 18B.

It is understood that the electrode assembly 354 may be assembled onto the tube according to various alternative and/or additional manners. For example, a clam shell construction that allows the electrode assembly 354 to be snaped together or fixed together (e.g., welding, adhesives) onto the ET tube body 60. Nevertheless, the electrode assembly 354 may be assembled on to the tube body 60 to provide the contacts for the electrodes of the ET tube 38.

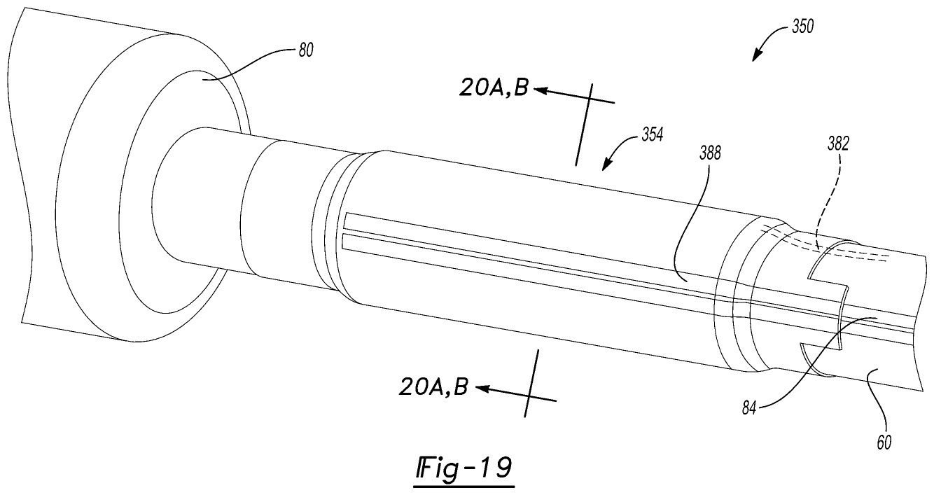

Turning reference to FIG. 19, the assembled ET tube 350 may include in the electrode assembly 354 positioned on the tube body 60. The assembled ET tube 350 may then be positioned within the subject 36, as illustrated in FIG. 1. The inflatable portion 80 may be inflated to assist in maintaining the ET tube 350 within the subject and sealing the passageway. Further, the bladder 378 of the electrode assembly 354 may be expanded to ensure appropriate contact of the contacts 388, 392 with a selected portions of the subject 36. Further, the expansion of the bladder 378 may allow for only a selected pressure to be applied to various portions of the subject 36, such as the vocal cords. Further, the contacts 388, 392 may be substantially flat and have little to no height or surface exterior to the exterior surface 374 of the sleeve 370. According to various embodiments, for example, the contacts 388, 392 may be printed on the electrode assembly 354.

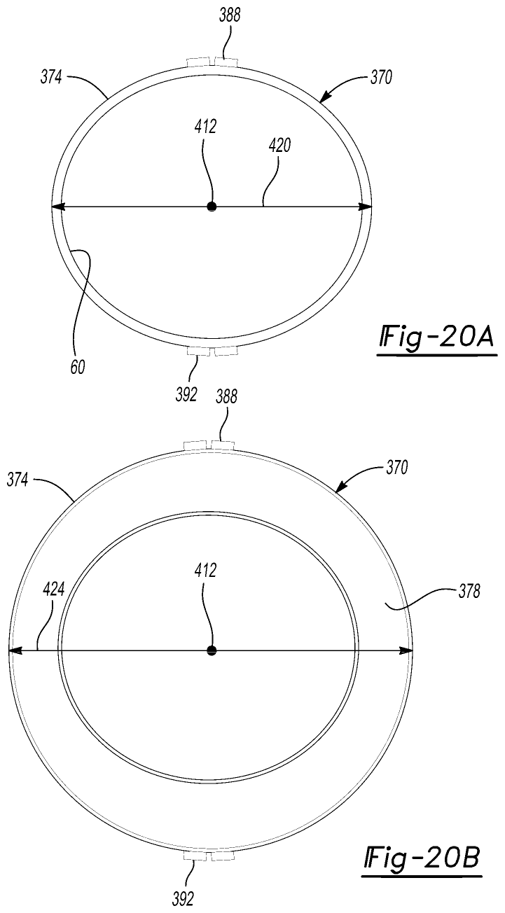

With continuing reference to FIG. 19 and additional reference to FIGS. 20A and 20B, the tube body 60 may have the sleeve 370 positioned thereon. In an uninflated or first configuration the exterior surface 374 of the sleeve 370 may have a first dimension 420, which may be a diameter, that is substantially equivalent to a diameter or external dimension of the tube 60.

At a selected time, the bladder 378 may be inflated to expand the exterior surface 374 of the sleeve 370 to have a second dimension 424 that is larger than the first dimension 420. The second dimension 424 may be any appropriate dimension and may be selected to move the electrodes 388, 392 into a selected contact with portions of the subject 36. It is understood that the amount of the inflation of the bladder 378 may be selected by the user 31 to achieve a selected pressure of the contact 388, 392 onto the subject.

The amount of pressure on the subject 36, however, may be selected and/or altered during a procedure based upon a selection by the user 31. Nevertheless, as the bladder 378 expands it radially expands the exterior surface 374 of the sleeve 370 and the electrode contacts 388, 392 may also move therewith. Thus, the contacts 388, 392 may be moved with the sleeve 370 to contact the subject 36 in a selected and/or appropriate manner. Further, as discussed above and illustrated in FIGS. 19 through 20B, the contacts 388, 392 may have an external dimension that is substantially similar to or equal to an external dimension or contour of the surface 374 of the sleeve 370. Therefore, a high point or pressure point may be substantially eliminated with the contact assembly 354.

The monitoring system 16 may include various contacts to contact to the subject 36, including those on the ET tube 38. Various other contacts, such as return contacts may also be connected to the subject 36. For example, as illustrated in FIG. 21, a skin or return electrode contact 440 may be positioned on a portion of the subject 36, such as on a skin surface. The contact 440 may be connected to via a connector or conductor 442 to the monitoring assembly 20. The connector 442 may include one or more traces such as a return trace 444 and a ground trace 448. Both of the traces may be connected with a single connector 450 to the monitoring assembly 20. According to various embodiments, the connector 442 may include or be connected to a wireless transmission assembly to transmit a wireless signal to the monitoring assembly 20 from the return electrode contact 440 assembly.

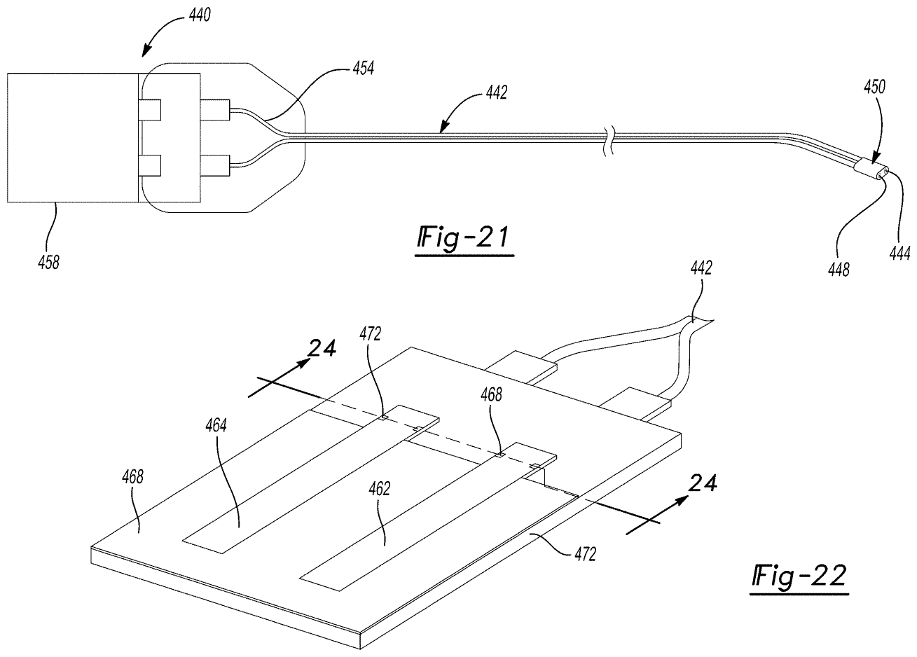

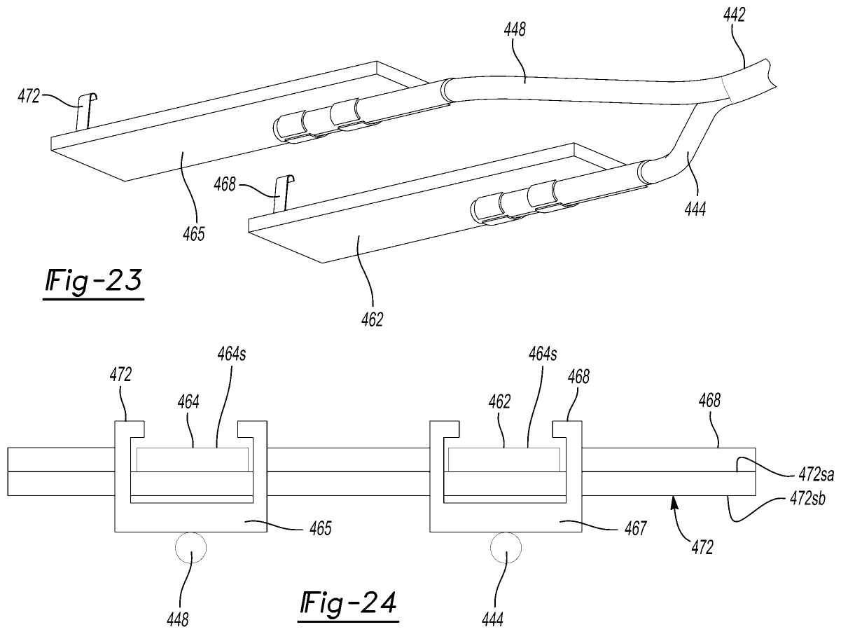

The return contact 440 may include various portions, as illustrated in FIGS. 21, 22, 23, and 24. As initially illustrated in FIG. 21 the surface contact 440 may be connected via the connectors 442 to the plug 450. The return contact 440 may include various portions such as an encased portion 454 and an adhesive portion that may be covered with a release tape 458. The release tape 458 may cover contacts including a first contact 462 and a second contact 464. The release tape 458 may cover an adhesive 468. The adhesive 468 may adhere the contact 440 to a surface of the subject 36, as discussed further herein. The adhesive 468 may be an appropriate adhesive such as 3M® 1504 Hi-Tack Transfer Adhesive. The adhesive 468 may also be referred to as a pressure adhesive.

The adhesive 468 may also be placed on the base 472 and allow the adhesive to contact the subject. The base 472 may be formed of a selected material that allows pressure to be applied to so that the adhesive 468 adheres to the subject 36. The base 472, therefore, may be formed of selected polymers or nonconductive materials. Appropriate polymers may include Polyethylene terephthalate (PET).

The contacts 462, 464 may be provided as separate members (e.g., foil or bar members formed of a conductive material) and be adhered to the base 472 in a selected manner. An adhesive, brazing, welding, or the like may be used to fix the contacts 462, 464 to the base 472. Thus, the contacts 462, 464 may be positioned on the subject 36 with the base 472. In various embodiments, the contacts 464, 464 may be formed on the base 472 of a conductive material, such as a conductive paint on the base 472. In various embodiments, the contacts 462, 464 may be formed as a conductive trace on the base 472. In this manner, the base 472 may operate as a printed circuit board and the contacts 462, 464 are traces formed thereon. This allows the contacts to be formed with the base 472 as opposed to being mounted or fixed thereto separately. This may increase longevity and/or reduce production and assembly costs and concerns.

The connectors 442 (including connections (e.g., traces or wires) 444, 448) may interconnect with the contacts 462, 464 by terminals or contact plates such as a first contact plate 465 and a second contact plate 467. Each of the contact plates may have prongs or extensions 468, 472 to engage the respective contacts 462, 464. The terminals 465, 467 may be engaged to the base 472 in any appropriate manner, such as via the prongs 468, 472 and other connection mechanisms. For example, the terminals 465, 467 may be fixed to the base 472 such as by welding, adhesives, brazing, or the like.

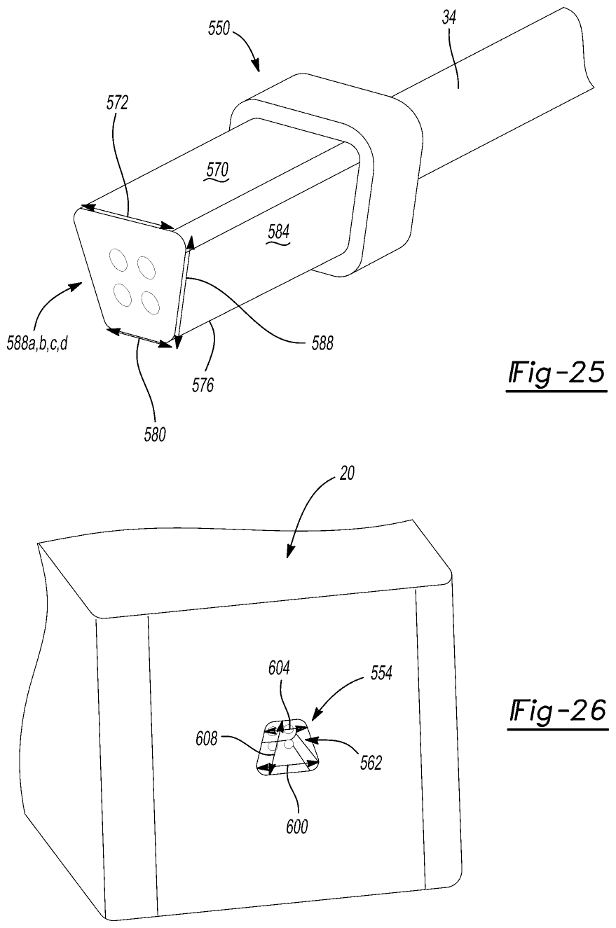

As illustrated in FIG. 24 the terminals 465, 467 may be fixed to a surface 480 of the base 472 and the prongs 468, 472 may extend through the base 472 to engage the contacts 460, 464. In this way the terminals 465, 467 may be fixed to the contacts 462, 464 which may be fixed to the base 472 and of the terminals may have an electrical connection with of the contacts 462, 464. The entire return electrode 440, therefore, may be substantially fixed member that may be affixed to the subject 36 in an appropriate manner. As a fixed member, the return electrode may be provided as a single unit that is applied to the subject as a single unit.

For example, as noted above, the release tape 458 may be removed from the adhesive 468 at an appropriate time by the user, such as the user 31. The return electrode 440 including the contacts 462, 464 may be adhered on an appropriate portion of the subject 36. The adhesive 468 may be a contact adhesive such that the user 31 presses the return electrode 440 onto the subject 36 to activate the adhesive. The electrode assembly 440 may then be adhered to the subject 36 for a selected period of time and act as a return electrode for the system 16. Thus, both of the contacts 462, 464 may contact the patient 36 substantially simultaneously when the return electrode 440 as applied to the subject 36. This may allow for the selected placement for performing a selected procedure.

Further, the contacts 462, 464 are or are essentially surface contacts and contact a surface of the subject 36. For example, the contacts 462, 464 contact a surface of a skin surface of the subject. The contacts 462, 464 do not and/or need not pierce the skin surface. Thus, the signal may be sensed by the monitoring system 16 and/or a ground may be provided and assist in the display with the monitoring assembly 20 without requiring insertion of electrodes into the subject 36. The signal may be sensed at the contacts 462, 464 without puncturing the skin of the subject 36. Similarly, removal of the return electrode 440 may only require pulling the electrode 440 from the subject 36. The contact adhesive may be efficiently removed by the user 31 from the subject 36 at an appropriate time. In various embodiments, the contacts 462, 464 may provide various features to the system 16. For example, the contact 462 may be a ground contact and the contact 464 may be a return contact. Thus, the two contacts 462, 464 may be separate and individual and provide only a single feature to the system, i.e., only a ground and/or only a return contact.

According to various embodiments, the contacts 462, 464 may both include substantially flat or planar surfaces 462s and 464s. The surfaces 462s, 464s may be substantially flat and/or smooth and contact the surface of the subject. Thus, the contact surfaces 462s, 464s need not pierce the subject 36. Further, the terminals 465, 467 may also have substantially flat surfaces. Further, both the contacts 462, 464 and the terminals 465, 467 may include substantially flat and/or smooth surfaces to engage respective first side 472sa and second side 472sb of the substate 472.

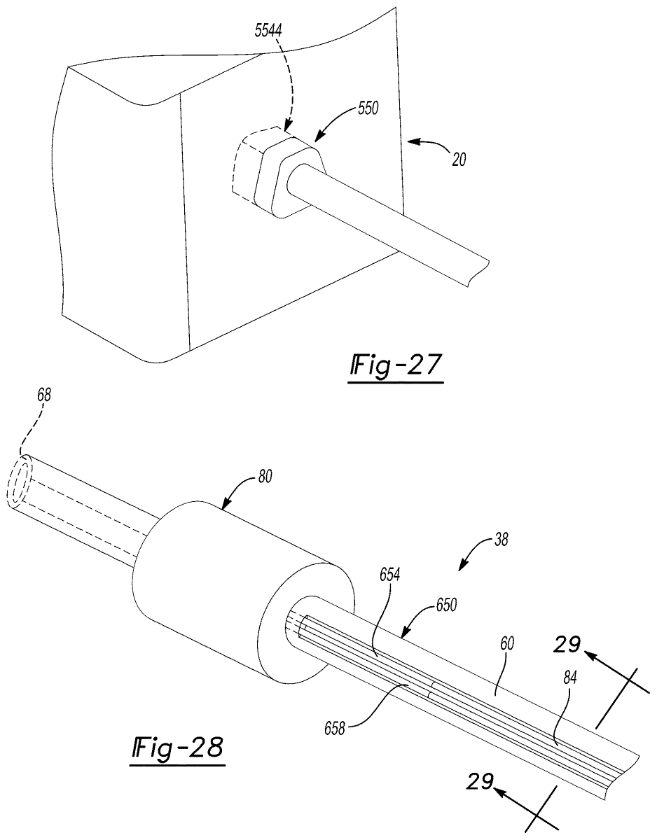

Various components may be connected to the monitoring assembly 20. As discussed above, various wireless protocols may be used. In addition, and/or alternatively, wired connections may be used to connect instruments to the monitoring assembly 20. With continuing reference to FIG. 1 and additional reference to FIGS. 25, 26, and 27, the connector 34 may have a plug or connector 550 that engages the monitoring assembly 20 in a socket 554. The connector 34 may include a plurality of leads, such as conductive wires, that extend through and/or to the connector 550. For example, the connector 34 may include for wires that may terminate in four connections at a connection region 558 of the connector 550. In various embodiments, for example, the collection portions 558 include for connection portions 558a, b, c, and d.

The connector 550 may have a selected geometry to limit engagement in the socket 554 to a selected manner. A unique configuration or geometry of the plug 550 may allow substantially only a single orientation for keyed insertion into the socket 554. Therefore, a user, such as the user 31, may efficiently connect the plug 552 to the socket 554 in an appropriate or selected orientation. The selected orientation may ensure that each of the connections in the connector 34 engage appropriate individual connections in the socket 554. As illustrated in FIG. 26, the socket 554 may have socket portions 562. Each of the plug portions 558 and the socket portions 562 may engage or allow for connection of selected portions on the ET tube 38. For example, the respective or multiple electrodes, sensors, or the like may be engaged to individual connections in that the monitoring apparatus 20 via the individual connections.

The plug 550 may have a selected geometry, such as a trapezoidal geometry. In various embodiments, therefore, the trapezoidal geometry may include a first side 570 having a first dimension 572, a second side 576 having a second dimension 580, a third side 584 having a third dimension 588. A fourth side of the plug 550 may include a dimension is similar to the dimension 588. The socket 554 may include sides have been dimensions substantially similar to the dimensions of the plug 550. For example, the dimensions of the sides of the socket 554 may have a tolerance of about 0.01 mm greater than the dimensions of the side of the plug 550. For example, the plug 554 may include a first dimension 600, a second dimension 604, and a third dimension 608. The dimension 600-608 may allow for a substantially tight or snap fit engagement with the plug 550. Thus, the plug 550 may be held in the socket 554 in an efficient manner and at a selected unique orientation achievable due to the geometry of the plug 550 and it and the respective stock at 554.

Additionally, the unique and cooperative shapes of the plug 550 and socket 554 may allow for an efficient identification of the socket 554 that is selected to engage or receive the plug 550. It is understood that other indications may also be provided, such as color coding, word identification, the like. Nevertheless, the cooperative or substantially equivalent shapes, such as mere shapes, may allow for efficient identification of the respective a plug and socket connection. It is understood, however, that other appropriate keyed shapes may be used that are generally not symmetrical or uniform about a perimeter, such as a projection may be included.

As illustrated in FIG. 1, the ET tube 38 includes various portions, such as the inflatable portion 80 near the distal end 68. Along the tube body 60 of the ET tube 38 are the connectors 84 and the electrodes 32. With reference to FIG. 28, an electrode 650 may be formed or provided as a portion of the connector 84 having insulation removed therefrom and bare wire or conductor material 654, 658 exposed on a surface of the body 60 of the ET tube 38.

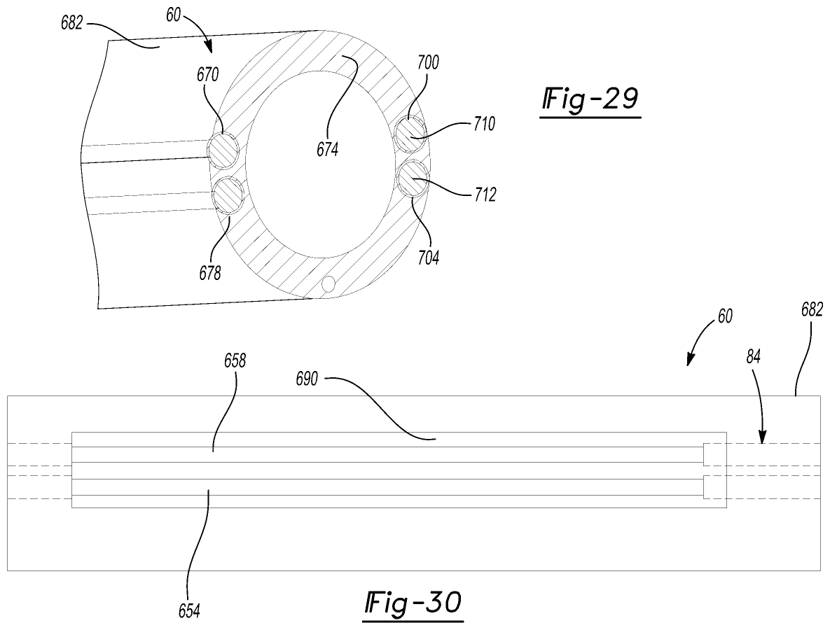

With continuing reference to FIG. 28 and additional reference to FIGS. 29 and 30, the connectors 84 may be formed and positioned in one or more grooves or surface depressions of the body 60. For example, as illustrated in FIG. 29, a groove or depression 670 may be formed in an external or surface wall 674 of the body 60. A second groove of 678 may also be formed in the body wall 674. Thus, the connectors 84 may be positioned within the respective grooves 670, 678 such that an external surface of the connectors 84 are generally coextensive with a surface 682 of the body 60 of the ET tube 38. Electrodes may be formed by removing a selected portion of an insulation of the connectors 84 and having the exposed regions at 654, 658. The exposed regions 654, 658 may be substantially coextensive or slightly above the surface 682 of the body 60. A depression or groove 690 may be formed in the body 60. The exposed portions 654, 658 may be adhered within the groove 690 in a selected manner, such as with an adhesive. Selected adhesives may include ultraviolet (UV) cured adhesives that may include materials that are cured with UV radiation.

The connectors 84 are formed or placed within the grooves 670, 678 and the exposed region 654, 658 are positioned within the groove 690. The grooves may allow the exterior dimension of the body 60 to be substantially uniform. Therefore, a raised portion or pressure point may be substantially eliminated in the ET tube 38. The body 60 of the ET tube 38 may be substantially uniform along a length of the body 60. It is further understood that a plurality of other connectors and/or electrodes may also be provided in the wall 670 of the tube 60. As illustrated in FIG. 29, additional grooves may be provided at any appropriate position, such as a first groove 700 and a second groove 704 may be formed in the wall 674a may have respective connectors 710, 712 positioned therein. Therefore, the ET tube 38 may have any selected number of electrodes for contact with selected portions of the subject 36.

Regardless, the electrodes need not be formed as separate members that are then connected to the connectors 84. The electrode contacts may be formed by removal of insulation or not positioning insulation over connectors on the body 60. In various embodiments, for example, the connectors 84 may be substantially bare or exposed conductive material and the grooves 670, 678 may be insulated (e.g., filled) with a selected material while maintaining the groove 690 substantially open or uninsulated. Thus, the electrodes may be formed substantially integrally within the connectors 84 and eliminating and/or not requiring a secondary or additional connection or part of an electrode to the connectors 84.

In various embodiments, the grooves 670, 678 may be formed in the wall 674 of the body 60 in a selected manner, such as by molding the body 60 to include the grooves 670, 678. Additional or alternative processes for forming the grooves may include milling or removing material to form the grooves 670, 678, or other appropriate methods. The method of forming the respective grooves 670, 678 may not be specific save that they are able to receive the connectors 84.

Further, the connector's 84 may be assembled into the grooves 670, 678 in any appropriate manner such as by a press fit, sliding along a length of the body 60, or other appropriate manner. Further, according to various embodiments, the connectors 84 may be molded into the walls 674 of the tube 60. Thereafter, the tube 60 including the connectors 84 may have the grooves 690 formed including removal of insulation or material from the connectors 84 and to expose the electrode contact portion 654, 658. Various milling or sanding processes may be used to remove material from around the electrode portion 654, 658 after the connectors 84 are formed in and/or with the tube 60.

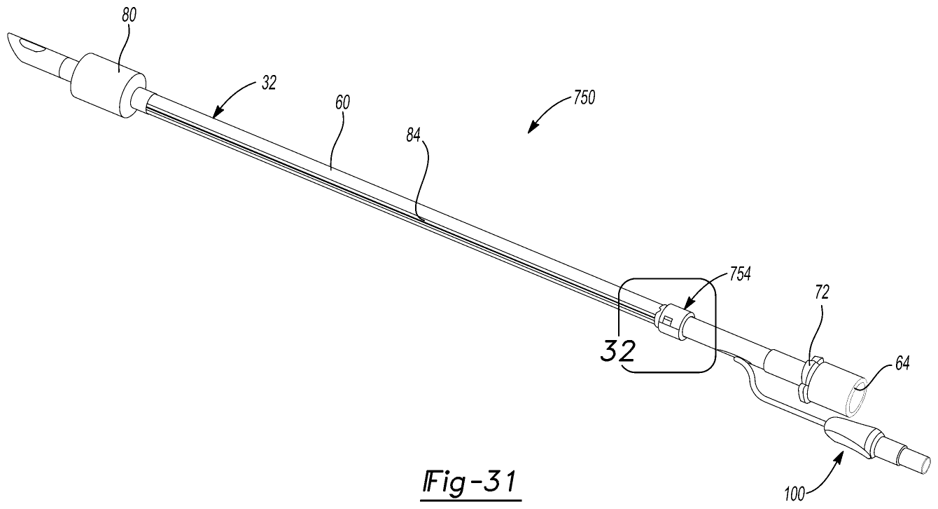

The ET tube 38 may be provided according to various embodiments, such as the ET tube 750 illustrated in FIG. 31. The ET tube 750 may include portions that are substantially similar to the ET tube, according to various embodiments, as discussed above. For example, the ET tube 750 may include the connector 72, the pilot balloon portion 100, the inflatable portion 80, and the body 60. As also discussed above, connectors may be positioned on and/or in the body 60 to connect the electrode region 32 of the ET tube 750 to the monitoring assembly 20. The connectors 84 may be connected to the monitoring assembly 20 according to various embodiments, such as with a connection module 754.

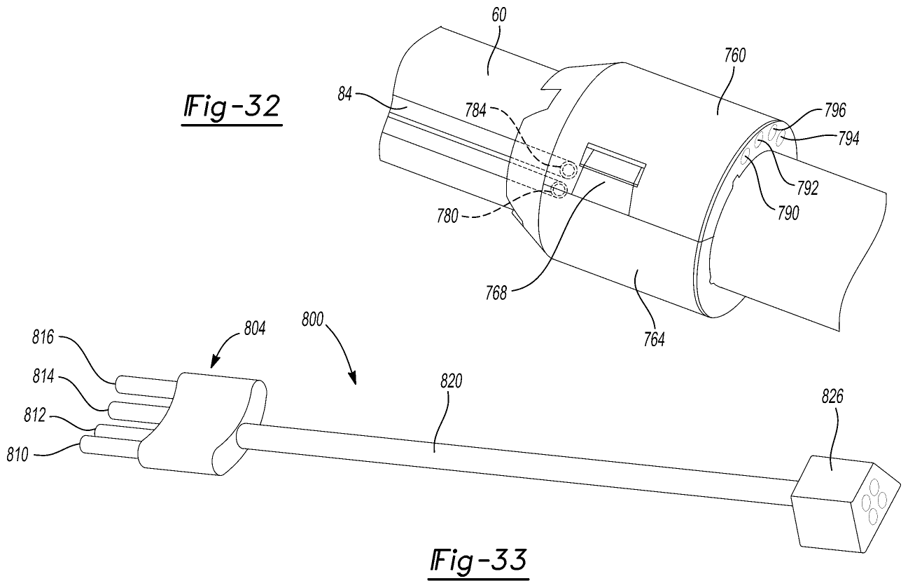

The connection module 754 may be positioned on the body 60 according to various embodiments, such as with clamshell configuration including at least a first body portion 760 and a second body portion 764, is illustrated in FIG. 32. A connector or latch 768 may be provided to fix the portions 760, 764 together. In various embodiments, for example, the connection module 754 may include a hinge portion (not illustrated) that allows the portions 760, 764 to hinge around the body 60. The latch 768 may latch or connect the two portions 760, 764 together to fix the connection module 754 to the body 60.

In various embodiments, the connector 754 may connect with the connectors 84. The connection with the connectors 84 may be in any appropriate manner, such as with pins 780, 784 that may pierce into the body 60 and engage the connectors 84. The piercing may occur when the clam shell 754 is fixed to the body 60. Other connections may include contacts within the connector 754 rather than pins 780, 784 to engage the connectors 84. It is further understood that the connector 754 may include wires that are connected with wires or connectors 84 over the ET tube 750, according to generally known techniques. The connectors 84 may be provided as wires placed or formed with the tube body 60, traces that are painted thereon or formed with a PCB. Thus, the connectors 84 may be any appropriate connection portion.

The connector 754 may include one or more sockets, such as four sockets 790, 792, 794, 796 that may allow connection to each of the traces on the ET tube 750, as discussed above. The sockets 790-796 may be engaged by one or more plugs of a plug or wire assembly 800. The wire assembly 800 may include a first or distal end 804 that may include a plurality of plugs, such as a first plug 810, a second plug 812, a third plug 814, and a fourth plug 816 to engage each of the respective sockets 790-796. The wire assembly 800 may include a main or single-wire assembly portion 820 that terminates in a plug 826. The plug 826 may be substantially similar or identical to the plug 550 discussed above, is illustrated in FIG. 25. The connectors or plug may also include separate or a plurality of plugs, as illustrated in FIG. 1.

Therefore, the connector 754 and the wire assembly 800 may allow for efficient connection of the ET tube 750 to the monitoring assembly 20 during use. The connector 754 may eliminate or reduce loose or dangling wires on the ET tube 750 and offer efficient connection of the monitoring assembly 20 to the ET tube 750. The connector 804 may have a selected geometry that mates in only a single orientation with the geometry of the connector 754. For example, as illustrated in FIGS. 32 and 33 the plug 804 may have a radius or curve and the connector 754 may have a radius or curve such that a unique orientation is required to connect the two. As noted above, the plug 826 may also have a unique geometry that mates with the monitoring assembly 20 in only a single manner or orientation. Thus, the proper or appropriate orientation and connections of the various contacts in wires may be made substantially efficiently by including the various connectors.

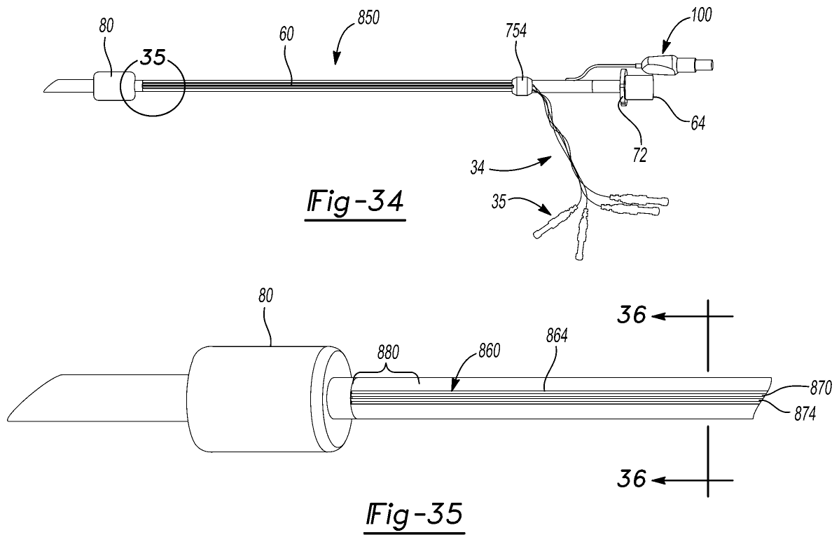

According to various embodiments, an ET tube 850 is illustrated in FIG. 34. The ET tube 150 may include various portions similar to those discussed above, including the connector 72, the pilot balloon 100, the inflatable portion 80, the body 60, and other portions, may be provided as connectors as discussed herein. Further, the connector assembly 754 may be provided, as discussed above, or interconnected with a plurality of individual wires and plugs similar to the connectors 34 as discussed above. The connector 754 may include a plurality of individual connectors and plugging portions 35 to interconnect with the monitoring assembly 20. Therefore, it is understood, that the connector 754 may be interconnected with the wire assembly 800 as discussed above and/or other countries, such as the connectors 34.

The ET tube 850 may include one or more connectors or traces such as in a conductor or trace assembly 860 they may be provided on a selected substrate 864 and formed as a printed conductive ink positioned on the substrates 864. The conductive ink may form a first trace 870 and a second trace 874 that are printed and cured on the substrate 864. The printed ink may be any appropriate printed conductive ink that is formed and cured on the substrate 864. The conductive ink traces may be connected to the connector 754 or with the connectors 34 in any appropriate manner. Nevertheless, the trace assembly 860 may be positioned on the body 60 of the ET 850 to allow for connection to the electrode portion 32 and the monitoring assembly 20.

The trace assembly 860 may define or form electrodes, such as an electrode region 880 by having a portion that is exposed and not covered by an insulator. The other portion of the trace assembly 860 may be covered with a selected insulator, including a portion of the tube body 60 and/or other insulation material. The traces may be formed as conductors in any appropriate manner on the substrate, such as by painting, printing, etching, coating, or the like.

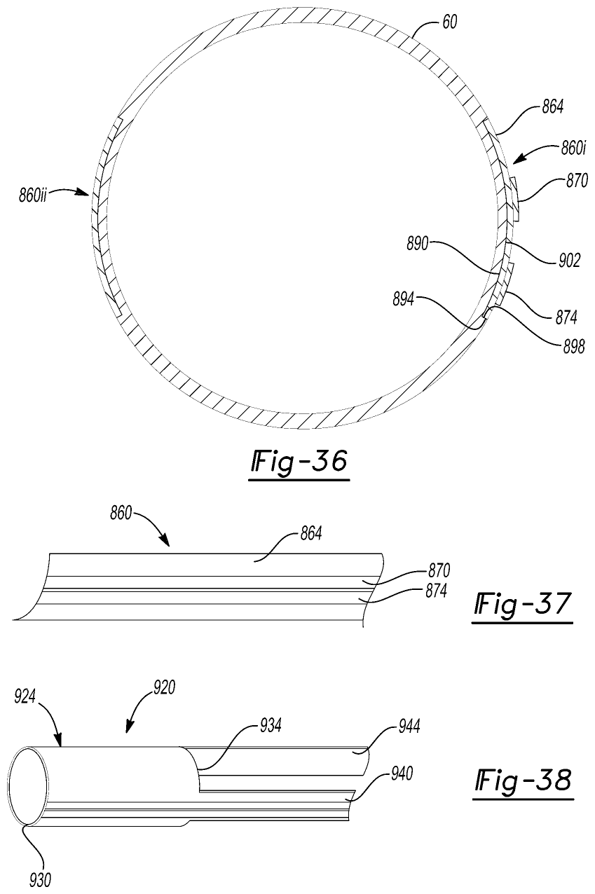

The trace assembly 860 may be assembled to the tube body 60 in any appropriate manner, such as being adhered to thereto with a selected adhesive. Turning reference to FIGS. 36, 37, and 38, the ET tube body 60 may include a groove portion formed with a bottom wall 890 and a sidewall 894. The bottom and sidewall 890, 894 may define a groove or depression to hold a portion of the conductor assembly 860. The conductor assembly 860 may, therefore, include a sidewall or surface 898 and a bottom wall or surface 902. The selected walls of the conductor assembly 860 may be affixed to the depression and any appropriate manner, such was with an appropriate adhesive as discussed above.

As illustrated in FIG. 36, a plurality of the conductive portions may be provided, such as those that are opposed to each other relative to the body 60. Therefore, the connector portions may include a first conductive portion 860i and a second conductive portion 860ii. The conductive portions 860i, 860ii may be formed in any appropriate manner. In various embodiments, the conductive assembly 860 may include the substrate 864 having formed or printed thereon the conductive material that form the traces 870, 874. The traces may be cured onto the substrate 864 at any appropriate time and the assembly may be fixed to the body 60.

Turning reference to FIG. 38, a unitary assembly may be formed as a conductive assembly 920. The unitary conductive assembly 920 may include a cylinder 924. The cylinder 924 may extend the length of the conduct of assembling or only a selected portion, such as between a first terminal end 930 and a second terminal end 934. If the cylindrical portion 924 is only a selected length of the conductive assembly 920, the traces extend from the cylindrical portion 924 as a first conductive trace portion 940 and a second conductive trace portion 944. Each of the conductive trace portions 940, 944 may include structure similar to the conductive trace portions 860, but may be formed with the cylindrical portion 924. As a single unit, the conductive assembly 920 may allow the conductive assembly 920 to be assembled to the tube body 60 as a single piece to assist in reducing manufacturing steps and pieces to be assembled.

Accordingly, the traces, such as the conductive traces 860, may be formed onto the substrate 864 at a selected time. The conductor assembly may then be fixed to the tube body 60 at an appropriate time. The tube body 60 may include various features, such as grooves or depressions to receive the conductive assembly 860. Thus, the conductive assembly 860 may include features such as a substantially thin or flat conductive member, including the traces 870, 874 without requiring the conductive portions to be printed directly onto the tube body 60.

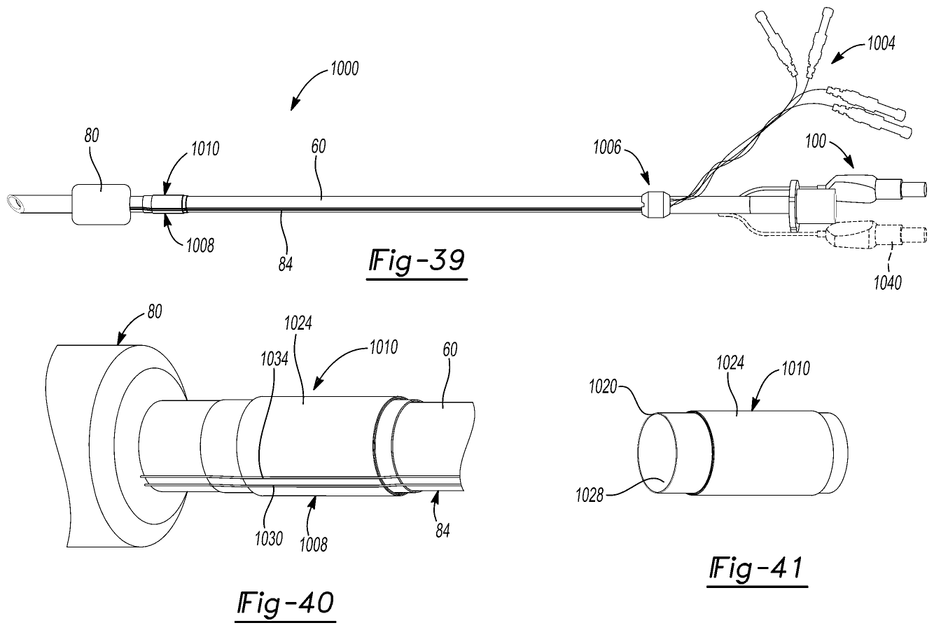

As discussed above, the ET tube 38 may be positioned within the subject 36 for various procedures. According to various embodiments, the ET tube may be provided as an ET tube 1000 as illustrated in FIG. 39. The ET tube 1000 may include various portions similar to those discussed above. For example, the ET tube 1000 may include the body 60 and connectors 84 that extend along the body 60 between connectors or plugs 1004 that may engage the monitoring assembly 20 and/or a connector or assembly 1006 that may be engaged on the body 60. Further, the inflatable portion 80 may be formed on the body 60 that may be inflated at a selected time to a selected amount such as through the pilot assembly 100.

The ET tube 1000 may further include electrodes 1008 that may be formed, such as with bare wire or exposed conductive material portions of the connectors 84. As discussed above, according to various embodiments, the electrode portions 32 may be provided to contact to the subject 36 to receive a signal from the subject 36. In various embodiments, the electrode portions 1008 may be positioned over a cushion or elastic portion 1010. The cushion portion 1010 may be positioned on the body 60 in any appropriate manner and may be formed of selected materials, as discussed herein.

The cushion portion 1010, according to various embodiments, may allow the electrode connections to move toward an external surface of the body 60 of the ET tube, according to various embodiments, when a pressure or force is applied to the electrode connections. That is, as a force is applied to the electrode connections the electrode connections may move into the cushion and/or the cushion may move to allow the electrode connections to move in the direction of the applied force. Thus, a counter force applied by the electrode connections may be less than the force applied to the electrode connections and/or the force may be dispersed by the cushion portion 1010.

With continuing reference to FIG. 39 and additional reference to FIG. 40, the cushion portion 1010 may be positioned on the body 60. With reference to FIG. 41 the cushion portion 1010 may be formed as a cylindrical member including a first support portion 1020 and a second or cushioning portion 1024. The support portion 1020 may be formed to have been internal surface 1028 that has a dimension that is similar to an exterior dimension of the body 60. Thus, the cushion assembly 1010 may be fit onto the body 60. In various embodiments additional materials or processes may be used to fix of the cushion assembly 1010 to the body 60, such as an adhesive, welding, or the like. For example, an adhesive material may be placed in the body 60. The cushion assembly may be slid over the adhesive. The adhesive may then be cured. The cushion assembly 1010 may then be fixed to the body 60.

Generally, the cushion assembly 1010 may be positioned on the body 60 prior to positioning the electrode portions 1008. The electrode portions 1008 may include exposed wire portions, such as a first wire portion 1030 and a second wire portion 1034. The wire portions 1030, 1034 may overlay the cushion portion 1024 of the cushion assembly 1010. The cushion portion 1024 may deflect when pressure is applied to the electrode portions 1030, 1034, such as by the subject 36. Therefore, a reduction of a pressure points may occur due to the cushion portion 1024 of the cushion assembly 1010. That is, pressure applied to the electrode portion 1008 may cause the electrode portion 1008 to be pushed into the cushion portion 1024. Thus, reducing a pressure experienced by the subject 36 or any portion thereof, such as the vocal cords of the subject 36.

The electrode portions 1030, 1034, however, may not be interrupted by the cushion assembly 1010. The electrode portions 1030, 1034 may overlay the cushion portion 1010 and/or contact the subject 36. Thus, the electrode portions 1030, 1034 may still receive a signal from the subject 36 and transmit it along the connectors 84 to the plugs 1004. The electrode portions 1030, 1034 may not exert a selected or greater than a selected pressure on the subject, however, due to the question portion 1024 the cushion assembly 1010. Generally, the cushion 1010 may be to add cushion to make the electrode adaptive and/or non-abrasive onto the tissue.

The electrode assemblies 1030, 1034, however, may be any appropriate electrode portions. As discussed above, the electrode portions 1030, 1034 may be provided as exposed electrical conductive material from a printed circuit board, such as a flexible printed circuit board (FPC), conductive material printed directly onto the ET tube 1000 which may also be printed on to the cushion assembly 1010, or other appropriate mechanism for forming the electrode portion 1008 including those discussed herein.

The cushion assembly 1010 may include various compositions or constructions. In various embodiments, for example, the cushion portion 1024 may be formed of a selected elastic material, such as an open cell foam, closed cell foam, rubber, Thermoplastic polyurethane (TPU), or silicone or the like. The cushion portion 1024 may have a fixed geometry (e.g., size) and/or may be selectable by the user 31.

In various embodiments, the cushion portion 1024 may also be formed as an inflatable bladder. The bladder may be filled with a selected material, such as a fluid, gel, etc. In various embodiments, for example, the bladder may be filled with air similar to the inflatable portion 80. The bladder may be formed over the base 1020 and positioned on that the body 60 of the ET tube 1000. The bladder may be filled prior to installation onto the body 60 or after installation onto the body 60.

For example, the bladder may be filled to a selected pressure and installed on the body 60. In various embodiments, similar to those discussed above, such as in the ET tube 350 illustrated in FIG. 18, the cushion portion 1024 may be inflated at a selected time, such as through an inflation assembly 1040. The inflation assembly 1040 may be similar to the pilot balloon assembly 100 but for filling of the cushion portion 1024 at a selected time and/or to a selected degree. Thus, the ET tube 1000 may be positioned in the subject and the cushion portion 1024 may be inflated to a selected pressure after the installation of the ET tube 1000 into the subject 36.

Regardless, the cushion portion 1024 of the cushion assembly 1010 may be provided to reduce pressure applied to or placed on the subject 36 of the electrode portions 1008 of the ET tube 1000, or in ET tube according to various embodiments including those discussed herein. The cushion assembly 1010 may be formed and/or assembled in any appropriate manner as discussed above. The cushion assembly 1010 may be provided to assist in minimizing or eliminating pressure applied to the subject due to the electrodes 1008.

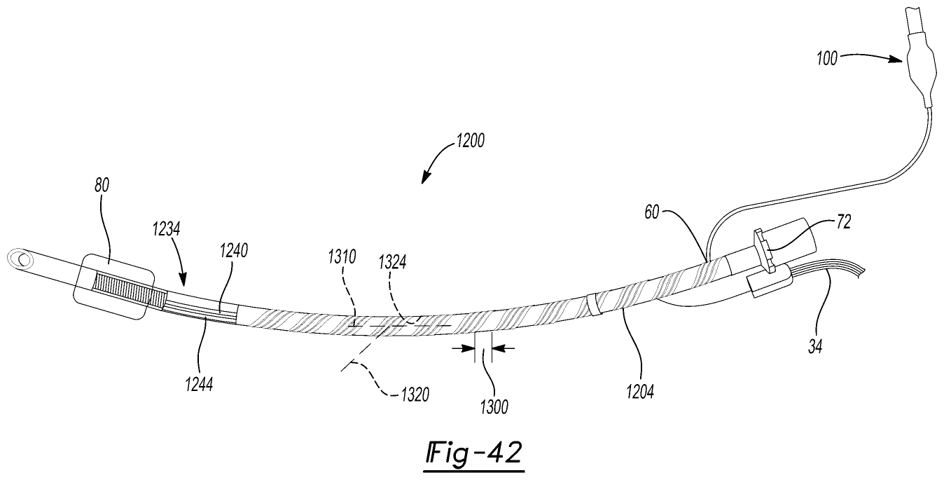

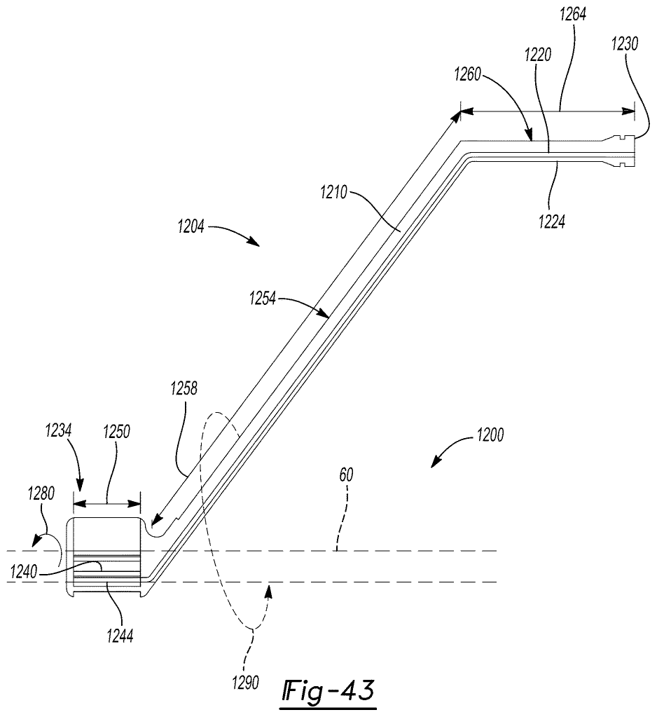

The ET tube 38, as discussed above may include various portions such as the contact region 32, inflatable portion 80, and other selected regions. With reference to FIGS. 42 and 43, an ET tube 1200 is illustrated. The ET tube 1200 may include portions similar to those discussed above, including the inflatable region 80, the connector portion 72, the pilot balloon portion 100, the body 60, and the connector portions 34. It is understood, however, that all of the portions are not required in various embodiments. The connector portion 34 may connect with a flexible printed circuit board (FPC) 1204. The FPC 1204 is illustrated alone in FIG. 43. The FPC 1204 may be placed on the body 60 of the ET tube 1200 in a helical or spun manner. In various embodiments, the FPC 1204 may include features similar to that disclosed in U.S. Pat. No. 9,226,689, incorporated herein by reference.

The FPC 1204 may include various portions, such as a substrate 1210 on which one or more conductive traces, such as a first conductive trace 1220 and a second conductive trace 1224. It is understood that more than two or any appropriate number of traces may be provided. The two traces 1220, 1224 are merely exemplary for the discussion herein.

The conductive traces 1220, 1224 may extend from a distal or connector end 1232 to an electrode and/or portion 1234. The connector portion 1230 maybe connected to the connectors 34, as illustrated in FIG. 42. The electrode portion 1234 may be wrapped around the body 60 of the ET tube 1200 and have exposed conductive portions to form the electrodes of the ET tube 1200. In various embodiments the electrode portion 1234 may include a first electrode 1240 and a second electrode 1244 that may interconnect with the respective traces 1220, 1224.

The electrodes 1240, 1244 and/or the traces 1220, 1224 may be formed on to the substrate 1210 and any appropriate manner. As discussed above, in various embodiments, the electrodes 1240, 1244 and/or the traces 1220, 1224 may be printed onto the substrate 1210 and cured thereon. According to various embodiments, electrodes 1240, 1244 and/or the traces 1220, 1224 may be formed as wires that are placed or fixed to the substrate 1210. According to various embodiments, electrodes 1240, 1244 and/or the traces 1220, 1224 may be formed as etched portions that are filled with a conductive material. Regardless of the formation process, the FPC 1204 includes the electrodes 1240, 1244 and/or the traces 1220, 1224. The FPC electrodes 1240, 1244 may be exposed only for a short distance, approximately 30 mm. For a left portion of conductive traces, they may be covered with dielectric ink.

Thus, the FPC 1204 may be formed as a construct that may be positioned onto the tube body 60 of the ET tube 1200. This allows the electrodes 1240, 1244 and/or the traces 1220, 1224 to be substantially flat when positioned on the body 60. That is, the electrodes 1240, 1244 and/or the traces 1220, 1224 extend only a short distance, such as less than 1 mm, or less than about 0.1 mm, or less than about 0.01 mm from the surface of the tube 60. Further the wrapping of the FPC 1204 on the body 60 may allow for a flex and/or movement of the body 60 in a selected manner while reducing a possibility of breaking the electrodes 1240, 1244 and/or the traces 1220, 1224 and reducing or minimizing a signal from the electrode contacts 1240, 1244. It is understood that any appropriate number of electrodes 1240, 1244 and/or the traces 1220, 1224 can be provided on the FPPC 1204.

The FPC 1204 may be formed in a selected manner, such as in the construct shape as illustrated in FIG. 43. The electrode contact portion 1234 may extend along a selected distance 1250. A first or intermediate portion 1254 may extend a second distance 1258 to a proximal portion 1260 near the connector 1230. The proximal portion 1260 may extend a third distance 1264. The various distances 1250, 1258, 1264 may be selected based upon a geometry, size, or the like of the body 60 of the ET tube 1200. For example, the length of the body 60, a diameter of the body 60, or other configurations of the body 60 may dictate selected sizes of the portions of the FPC 1204. Nevertheless, the contact portion 1234 may be selected to include an appropriate geometry and surface area for contact with of the subject 36 to ensure a proper signal therefrom. Further the intermediate portion 1254 may be selected to ensure that the connectors 1220, 1224 extended appropriate a length along the body 60 of the ET tube 1200.

In various embodiments, the electrode to contact portion 1234 may be wrapped around the ET tube body 60, such as generally the direction of arrow 1280. The electrode contact portion 1234 may generally form a cylinder to extend around the tube body 60. The intermediate portion 1254 may be wrapped in a substantially cylindrical manner generally the direction of arrow 1290. The intermediate portion 1254 may, therefore, include a spacing between wraps, such as a selected dimension spacing 1300. Further, the helical wrapping may cause angles to be formed between the wraps and an axis 1310 of the body 60. As illustrated in FIG. 42, for example, the body 60 may include the axis 1310. The wrapping may occur along a wrapping axis or line 1320. An angle 1324 may be defined or formed between the axis 1310 of the body 60 and the axis of wrapping 1320. The angle 1324 may be a selected angle such as about 10° to about 80°, including about 40° to about 60°, and further including about 50° to about 57°. The number of wraps may be based upon a length of the ET tube 1200.

Further the FPC 1204 may be affixed to exterior surface of the body 60 in a selected manner. For example, various adhesives may be provided to adhere to the FPC 1204 to the body 60. Selected adhesives may include the 3M F9465PC.

The FPC 1204 may include a selected dimension to assist in positioning of the electrode contacts 1244, 1246 on the body 60 without substantially increasing a diameter geometry of the body 60. Similarly, the connector is on the FPC 1204 may also be substantially flat and not include a dimension that is substantially greater than the exterior geometry or dimension of the ET tube 60. For example, not increasing a diameter by more than 1 mm, or more than about 0.1 mm, or more than about 0.01 mm. Thus the pressure on the subject 36 may be minimized to a selected degree to reduce strain placed on the subject 36.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C #, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

1. A monitoring system, comprising:
an elongated tube having a first end and a second end, wherein the tube includes a wall defining an internal surface and an external surface; and
an electrode contact and connector assembly, comprising,
a substrate configured to be positioned on the external surface,
an electrode contact printed on the substrate with a conductive material, wherein the electrode contact is exposed to a subject passage from the external surface of the tube,
a connector printed on the substrate with the conductive material, wherein the connector is configured to extend from the electrode contact to the second end;
wherein the substrate is fixed to the external surface as the electrode contact and connector assembly;
wherein the internal surface of the elongated tube defines a cannula.

2. The system of claim 1, wherein the external surface includes a groove formed therein, wherein the groove includes an internal surface within an external dimension of a portion of the external surface separate from the groove;
wherein the electrode contact and connector assembly is fixed to the elongated tube within the groove.

3. The system of claim 1, wherein the substrate includes a flexible substrate configured to at least flex with the elongated tube;
wherein the electrode contact and the connector are printed onto the substrate;
wherein the substrate and the electrode contact and the connector are flexible to at least flex with the elongated tube.

4. The system of claim 3, wherein the substrate and the electrode contact and the connector are a single unit fixed to the elongated tube.

5. The system of claim 1, further comprising:
a cushion portion, wherein the cushion portion is positioned between at least the electrode contact and the external surface.

6. The system of claim 5, wherein the cushion portion is formed of a compressible foam.

7. The system of claim 5, further comprising:
an inflation passage;
wherein the cushion portion comprises an inflatable bladder configured to be inflated via the inflation passage.

8. The system of claim 5, wherein the cushion portion is fixed to the elongated tube as a single member.

9. The system of claim 1, further comprising:
an inflatable member nearer the first end than the second end, wherein the inflatable member is configured to selectively seal a passage between the elongated tube and the subject passage;
an inflation passage configured to allow a fluid to pass from near the second end to the inflatable member; and
a pilot assembly connected to the inflation passage and configured to provide at least one of a visual, haptic or signal regarding an inflation status of the inflatable member.

10. A monitoring system, comprising:
an elongated tube having a first end and a second end, wherein the tube includes a wall defining an internal surface and an external surface;
an electrode contact assembly comprising (i) a first substrate and (ii) an electrode contact printed on the substrate with a conductive material, wherein the electrode contact is exposed to a subject passage from the external surface of the tube,
a connector assembly comprising (i) a second substrate configured to be positioned on the external surface and (ii) a connector printed on the substrate with the conductive material, wherein the connector is configured to extend from the electrode contact to the second end;
a cushion assembly, wherein the cushion portion is positioned between at least the electrode contact and the external surface; and
an inflatable member nearer the first end than the second end, wherein the inflatable member is configured to selectively seal a passage between the elongated tube and the subject passage;
wherein the substrate is fixed to the external surface as the electrode contact and connector assembly;
wherein the internal surface of the elongated tube defines a cannula.

11. The system of claim 10, wherein the electrode contact is printed on the cushion assembly.

12. The system of claim 11, wherein the cushion portion is formed of a compressible foam.

13. The system of claim 11, further comprising:
a cushion inflation passage;
wherein the cushion portion comprises an inflatable bladder configured to be inflated via the inflation passage.

14. The system of claim 13, further comprising:
an inflation member passage configured to allow a fluid to pass from near the second end to the inflatable member.

15. The system of claim 10, wherein the external surface includes a groove formed therein, wherein the groove includes an internal surface within an external dimension of a portion of the external surface separate from the groove;
wherein the electrode contact and connector assembly is fixed to the elongated tube within the groove.

16. The system of claim 10, wherein the first substrate includes a flexible substrate configured to at least flex with at least one of the cushion portion or the elongated tube.

17. A method of providing a tube for a monitoring system, comprising:
providing an elongated tube having a wall defining an internal surface and an external surface that extends from a first end to a second end;
providing an electrode contact assembly comprising printing on a first substrate an electrode contact of a conductive material;
providing the electrode contact exposed from the external surface of the tube;
providing a connector assembly comprising printing on a second substrate a connector of the conductive material;
connecting the connector and the electrode contact;
positioning a cushion assembly between at least the electrode contact and the external surface;
providing an inflatable member nearer the first end than the second end; and
fixing at least a portion of the substrate to the external surface;
wherein the internal surface of the elongated tube defines a cannula.

18. The method of claim 17, wherein providing the electrode contact assembly comprising printing on the first substrate the electrode contact of the conductive material comprises printing the electrode contact on the cushion assembly;
wherein the first substrate is separate from the second substrate.

19. The method of claim 18, further comprising:
assembling the cushion assembly onto the provided elongated tube to allow the connecting the connector and the electrode contact.

20. The method of claim 17, further comprising:
forming the cushion assembly to include a cushion portion operable to allow the electrode contact to move toward the external surface upon the application of an external force.
